SEQUENCE LISTING (19 sequences)

SEQ ID NO: 1 - protein, 400 aa, Homo sapiens [sequence omitted]

SEQ ID NO: 2 - protein, 347 aa, Homo sapiens
RAAAERGAVELKKNEFQGELEKQREQLDKIQSSHNFQLESVNKLYQDEKAVLVNNITTGERLIRVLQDQLKTLQRNYGRLQQDVLQFQKNQTNLERKFSYDLSQCINQMKEVKEQCEERIEEVTKKGNEAVASRDLSENNDQRQQLQALSEPQPRLQAAGLPHTEVPQGKGNVLGNSKSQTPAPSSEVVLDSKRRVEKEETNEIQVVNEEPQRDRLPQEPGREQVVEDRPVGGRGFGGAGELGQTPQVQAALSVSQENPEMEGPERDQLVIPDGQEEEQEAAGEGRNQQKLRGEDDYNMDENEAESETDKQAALAGNDRNIDVFNVEDQKRDTINLLDQREKRNHTL

SEQ ID NO: 3 - protein, 23 aa, Homo sapiens
SPPLVLAALVACIIVLGFNYWIA

SEQ ID NO: 4 - protein, 366 aa, Homo sapiens
SSRSVDLQTRIMELEGRVRRAAAERGAVELKKNEFQGELEKQREQLDKIQSSHNFQLESVNKLYQDEKAVLVNNITTGERLIRVLQDQLKTLQRNYGRLQQDVLQFQKNQTNLERKFSYDLSQCINQMKEVKEQCEERIEEVTKKGNEAVASRDLSENNDQRQQLQALSEPQPRLQAAGLPHTEVPQGKGNVLGNSKSQTPAPSSEVVLDSKRRVEKEETNEIQVVNEEPQRDRLPQEPGREQVVEDRPVGGRGFGGAGELGQTPQVQAALSVSQENPEMEGPERDQLVIPDGQEEEQEAAGEGRNQQKLRGEDDYNMDENEAESETDKQAALAGNDRNIDVFNVEDQKRDTINLLDQREKRNHTL

SEQ ID NO: 5 - protein, 169 aa, Homo sapiens
SSRSVDLQTRIMELEGRVRRAAAERGAVELKKNEFQGELEKQREQLDKIQSSHNFQLESVNKLYQDEKAVLVNNITTGERLIRVLQDQLKTLQRNYGRLQQDVLQFQKNQTNLERKFSYDLSQCINQMKEVKEQCEERIEEVTKKGNEAVASRDLSENNDQRQQLQALS

SEQ ID NO: 6 - protein, 150 aa, Homo sapiens
RAAAERGAVELKKNEFQGELEKQREQLDKIQSSHNFQLESVNKLYQDEKAVLVNNITTGERLIRVLQDQLKTLQRNYGRLQQDVLQFQKNQTNLERKFSYDLSQCINQMKEVKEQCEERIEEVTKKGNEAVASRDLSENNDQRQQLQALS

SEQ ID NO: 7 - protein, 37 aa, Homo sapiens
RAAAERGAVELKKNEFQGELEKQREQLDKIQSSHNFQ

SEQ ID NO: 8 - protein, 40 aa, Homo sapiens
LESVNKLYQDEKAVLVNNITTGERLIRVLQDQLKTLQRNY

SEQ ID NO: 9 - protein, 40 aa, Homo sapiens
GRLQQDVLQFQKNQTNLERKFSYDLSQCINQMKEVKEQCE

SEQ ID NO: 10 - protein, 40 aa, Homo sapiens
ERIEEVTKKGNEAVASRDLSENNDQRQQLQALSEPQPRLQ

SEQ ID NO: 11 - protein, 40 aa, Homo sapiens
AAGLPHTEVPQGKGNVLGNSKSQTPAPSSEVVLDSKRRVE

SEQ ID NO: 12 - protein, 40 aa, Homo sapiens
KEETNEIQVVNEEPQRDRLPQEPGREQVVEDRPVGGRGFG

SEQ ID NO: 13 - protein, 40 aa, Homo sapiens
GAGELGQTPQVQAALSVSQENPEMEGPERDQLVIPDGQEE

SEQ ID NO: 14 - protein, 40 aa, Homo sapiens
EQEAAGEGRNQQKLRGEDDYNMDENEAESETDKQAALAGN

SEQ ID NO: 15 - protein, 30 aa, Homo sapiens
DRNIDVFNVEDQKRDTINLLDQREKRNHTL

SEQ ID NO: 16 - DNA, 1203 bp, Homo sapiens
atgggcttgggaaacgggcgtcgcagcatgaagtcgccgcccctcgtgctggccgccctggtggcctgcatcatcgtcttgggcttcaactactggattgcgagctcccggagcgtggacctccagacacggatcatggagctggaaggcagggtccgcagggcggctgcagagagaggcgccgtggagctgaagaagaacgagttccagggagagctggagaagcagcgggagcagcttgacaaaatccagtccagccacaacttccagctggagagcgtcaacaagctgtaccaggacgaaaaggcggttttggtgaataacatcaccacaggtgagaggctcatccgagtgctgcaagaccagttaaagaccctgcagaggaattacggcaggctgcagcaggatgtcctccagtttcagaagaaccagaccaacctggagaggaagttctcctacgacctgagccagtgcatcaatcagatgaaggaggtgaaggaacagtgtgaggagcgaatagaagaggtcaccaaaaaggggaatgaagctgtagcttccagagacctgagtgaaaacaacgaccagagacagcagctccaagccctcagtgagcctcagcccaggctgcaggcagcaggcctgccacacacagaggtgccacaagggaagggaaacgtgcttggtaacagcaagtcccagacaccagcccccagttccgaagtggttttggattcaaagagacgagttgagaaagaggaaaccaatgagatccaggtggtgaatgaggagcctcagagggacaggctgccgcaggagccaggccgggagcaggtggtggaagacagacctgtaggtggaagaggcttcgggggagccggagaactgggccagaccccacaggtgcaggctgccctgtcagtgagccaggaaaatccagagatggagggccctgagcgagaccagcttgtcatccccgacggacaggaggaggagcaggaagctgccggggaagggagaaaccagcagaaactgagaggagaagatgactacaacatggatgaaaatgaagcagaatctgagacagacaagcaagcagccctggcagggaatgacagaaacatagatgtttttaatgttgaagatcagaaaagagacaccataaatttacttgatcagcgtgaaaagcggaatcatacactttaa

SEQ ID NO: 17 - protein, 401 aa, Homo sapiens
MMGLGNGRRSMKSPPLVLAALVACIIVLGFNYWIASSRSVDLQTRIMELEGRVRRAAAERGAVELKKNEFQGELEKQREQLDKIQSSHNFQLESVNKLYQDEKAVLVNNITTGERLIRVLQDQLKTLQRNYGRLQQDVLQFQKNQTNLERKFSYDLSQCINQMKEVKEQCEERIEEVTKKGNEAVASRDLSENNDQRQQLQALSEPQPRLQAAGLPHTEVPQGKGNVLGNSKSQTPAPSSEVVLDSKRQVEKEETNEIQVVNEEPQRDRLPQEPGREQVVEDRPVGGRGFGGAGELGQTPQVQAALSVSQENPEMEGPERDQLVIPDGQEEEQEAAGEGRNQQKLRGEDDYNMDENEAESETDKQAALAGNDRNIDVFNVEDQKRDTINLLDQREKRNHTL

SEQ ID NO: 18 - DNA, 1429 bp, Homo sapiens
gctcgaggccggcggcggcgggagagcgacccgggcggcctcgtagcggggccccggatccccgagtggcggccggagcctcgaaaagagattctcagcgctgattttgagatgatgggcttgggaaacgggcgtcgcagcatgaagtcgccgcccctcgtgctggccgccctggtggcctgcatcatcgtcttgggcttcaactactggattgcgagctcccggagcgtggacctccagacacggatcatggagctggaaggcagggtccgcagggcggctgcagagagaggcgccgtggagctgaagaagaacgagttccagggagagctggagaagcagcgggagcagcttgacaaaatccagtccagccacaacttccagctggagagcgtcaacaagctgtaccaggacgaaaaggcggttttggtgaataacatcaccacaggtgagaggctcatccgagtgctgcaagaccagttaaagaccctgcagaggaattacggcaggctgcagcaggatgtcctccagtttcagaagaaccagaccaacctggagaggaagttctcctacgacctgagccagtgcatcaatcagatgaaggaggtgaaggaacagtgtgaggagcgaatagaagaggtcaccaaaaaggggaatgaagctgtagcttccagagacctgagtgaaaacaacgaccagagacagcagctccaagccctcagtgagcctcagcccaggctgcaggcagcaggcctgccacacacagaggtgccacaagggaagggaaacgtgcttggtaacagcaagtcccagacaccagcccccagttccgaagtggttttggattcaaagagacaagttgagaaagaggaaaccaatgagatccaggtggtgaatgaggagcctcagagggacaggctgccgcaggagccaggccgggagcaggtggtggaagacagacctgtaggtggaagaggcttcgggggagccggagaactgggccagaccccacaggtgcaggctgccctgtcagtgagccaggaaaatccagagatggagggccctgagcgagaccagcttgtcatccccgacggacaggaggaggagcaggaagctgccggggaagggagaaaccagcagaaactgagaggagaagatgactacaacatggatgaaaatgaagcagaatctgagacagacaagcaagcagccctggcagggaatgacagaaacatagatgtttttaatgttgaagatcagaaaagagacaccataaatttacttgatcagcgtgaaaagcggaatcatacactctgaattgaactggaatcacatatttcacaacagggccgaagagatgactataaaatgttcatgagggactgaatactgaaaactgtgaaatgtactaaataaaatgtacatctga

SEQ ID NO: 19 - RNA, 21 nt, Artificial Sequence
  A synthetic siRNA
accucuuguggcuuugcuann

(19) United States
(12) Patent Application Publication
(10) Pub. No.: US 2014/0147437 A1
(43) Pub. Date: May 29, 2014

(54) METHODS TO ENHANCE CELL-MEDIATED IMMUNITY

(71) Applicant: Xiaojing Ma, Fort Lee, NJ (US)

(72) Inventor: Xiaojing Ma, Fort Lee, NJ (US)

(73) Assignee: Cornell Universtiy, Ithaca, NY (US)

(21) Appl. No.: 13/985,697

(22) PCT Filed: Feb. 16, 2012
(86) PCT No.: PCT/US12/25492

§ 371 Date: Jan. 6, 2014

Related U.S. Application Data
(60) Provisional application No. 61/443,569, filed on Feb. 16, 2011.

(51) Int. Cl. C07K 16/18; C12N 15/113

(52) U.S. Cl. CPC C07K 16/18; C12N 15/113
USPC 424/133.1; 424/139.1; 514/44 A

(57) ABSTRACT

This disclosure provides a method for enhancing cell-mediated immunity in individuals with disorders such as cancer or infection that involves administering an inhibitor of GOLPH2 to the individuals. For example, inhibition of GOLPH2 increases the endogenous production of IL-12.

This application claims benefit of the filing date of U. S. Provisional Patent Application No. 61/443,569, filed Feb. 16, 2011, the contents of which are specifically incorporated herein in their entirety.

### BACKGROUND

Cancer is a serious disease and a major killer. Although there have been advances in the diagnosis and treatment of certain cancers in recent years, there is still a need for improvements in diagnosis and treatment. Similarly, while treatment of viral and bacterial infections has improved over the last 10-30 years, there remains a need for new methods and compositions that can significantly improve the survival rate and/or lessen the duration of the infection.

Compositions and methods for stimulating the patient's own immune system may be helpful for treating a variety of diseases, including cancer as well as bacterial and viral infections. Some studies indicate that IL-12 may be able to activate the host's immune apparatus against a variety of tumors in animal models (see, Trinchieri & Scott (Curr Top Microbiol Immunol 238, 57-78 (1999); Rook et al., Blood 94, 902-8. (1999); Rook et al., Ann N Y Acad Sci 941, 177-84. (2001)).

Additional methods for modulating the immune system would be useful in a variety of treatment regimens for numerous diseases and disorders.

### SUMMARY

This disclosure provides a method to enhance the cell-mediated immunity of a mammal by administering an inhibitor of GOLPH2. Such inhibitors can enhance cell-mediated immunity of a mammal in a variety of ways including, for example, by increasing the endogenous production of interleukin-12 and/or interferon-γ. Thus methods and compositions for inhibiting GOLPH2 have utility in immunotherapy for cancers and for pathogenic infections that would benefit from cell-mediated immune responses for the control, amelioration and elimination of the disease, as well as for long-term protection against the disease or its recurrence. Diseases and disorders that may be treated by inhibiting GOLPH2 include, for example, cancers of the liver, prostate, lung, testes, pancreas and B-cells as well as various infectious diseases such as HIV/AIDS and hepatitis.

### BRIEF DESCRIPTION OF THE DRAWINGS

FIG. 1A-B illustrates the activities of BDSF^{IL-12} and its identification with GOLPH2. FIG. 1A shows that dendritic cells secrete a factor that inhibits interferon-γ secretion by activated T cells. The factor was termed BDSF^{IL-12}. T lymphocytes were isolated from C57BL/6 mouse spleen by CD4⁺ T cell MACS isolation kit, and were cultured for 4 days in RPMI medium (15% FBS, 20 ng/ml mIL-2). The T cells were then plated at 1×10⁶ cells/well in 1 ml, and stimulated with concanavalin A (ConA) at 5 μg/ml for 24 h in the presence or absence of culture supernatant from myeloid dendritic cells (500 μl). In particular, aliquots of these T cells were subjected to one of four treatments. Treatment type 1: addition of dendritic cell culture supernatant to the T cells, where the dendritic cells were resting and had not been stimulated. Treatment type 2: addition of dendritic cell culture supernatant to the T cells, where the dendritic cells had been stimulated with lipopolysaccharide (LPS). Treatment type 3: addition of dendritic cell culture supernatant to the T cells, where the dendritic cells were cultured with 2E2 supernatant (containing BDSF^{IL-12}). Treatment type 4: addition of dendritic cell culture supernatant to the T cells, where the dendritic cells were cultured with 2E2 supernatant (containing BDSF^{IL-12}) and were stimulated with lipopolysaccharide (LPS). FIG. 1B shows fractionation by SDS-PAGE of cell-free culture supernatants from resting and LPS-stimulated RAMOS cells as well as 2E2 cells, with detection of a major protein band named BDSF^{IL-12} (molecular weight>50 kDa). Preliminary characterization of BDSF^{IL-12} revealed the following biochemical properties: it is heat resistant; it is protease- and lipase-insensitive; it is produced by transformed B cells cultured without serum; and by size fractionation, BDSF^{IL-12} appeared to be >50 kDa. For further identification, cell-free culture supernatants from resting and LPS-stimulated RAMOS cells cultured in the absence of fetal bovine serum for 24 h (lanes 1-2) and that of 2E2 (lane 3) were boiled for 30 min, followed by trypsin treatment (50 ng/ml) for 30 min, and fractionation through an SDS-PAGE gel. The lower bands from both resting and LPS-stimulated RAMOS cell supernatants, which are identified by the lower arrow in FIG. 1B, were excised and analyzed by mass spectrometry. The reason for choosing the samples from RAMOS instead of 2E2 for final analysis was for its comparability of the stimulated sample with high BDSF^{IL-12} activity versus the unstimulated sample with low or little activity. Two proteins were identified in LPS-stimulated RAMOS supernatant as potentially corresponding to BDSF^{IL-12} by their significantly high scores over the control sample (resting RAMOS supernatant): one major and one minor, Golgi phosphoprotein 2 (GOLPH2; a major product) and Roquin (a minor product) with 7% and 1% coverage, respectively.

FIG. 2A-F show that the cellular location of GOLPH2 varies depending on the cell type as detected by FACS analysis and illustrate that GOLPH2 is secreted into the supernatant of different cultured cell lines. FIG. 2A-E shows that GOLPH2 is expressed abundantly intracellularly, and on the cell surface of both resting (FIG. 2A) and LPS-activated (FIG. 2B) primary human peripheral blood B lymphocytes. However, in the human hepatocellular carcinoma line HepG2, GOLPH2 is expressed more intracellularly than at the cell surface (FIG. 2C). In RAW264.7 cells (mouse macrophage cells), GOLPH2 expression appears entirely intracellular, and addition of LPS had little, if any, effect upon the level and locale of GOLPH2 expression (FIG. 2D). FIG. 2F shows a Western blot of cell culture supernatants from RAMOS cells (resting and LPS-activated, lanes 2-3, respectively), 2E2 cells (lane 4), HepG2 cells (human hepatocellular carcinoma (HCC), lane 5), B16 cells (mouse melanoma, lane 6), 4T1 cells (mouse mammary adenocarcinoma, lane 7), and RAW264.7 cells (mouse macrophage, lane 8). Recombinant human GOLPH2 expressed from a histidine-tagged expression vector was used as a positive control (lane 9). Unless otherwise indicated, resting cells were analyzed.

FIG. 3A-D are graphs illustrating some of the activities of BDSF^{IL-12}/GOLPH2. FIG. 3A is a graph illustrating interferon-γ secretion by activated T cells that were exposed to cell culture supernatants from various cell types. Human embryonic kidney 293 (HEK293) cells were transiently transfected with a vector expressing histidine-tagged human GOLPH2, or an unrelated nuclear protein, SREBP2. Forty-eight hours after transfection, cell-free culture supernatant was collected, and added to the dendritic cell and T cell cocultures in the same manner described for FIG. 1A above. Bar a: cell culture supernatant from unstimulated HEK cells; Bar b: cell culture supernatant from LPS-stimulated cells; Bar c: cell culture supernatant from SREBP-transfected cells; Bar d: cell culture supernatant from GOLPH2-transfected cells; and Bar e: cell culture supernatant from 2E2 cells. The small panel below the bar graph shows a western blot of HEK293 cell supernatant probed with histidine tag monoclonal antibodies, after the cells were recombinantly transfected with SREBP2 (bar c) or GOLPH2 (bar d). As shown, GOLPH2 was expressed and secreted into the supernatant used for the results shown in bar d but not into the supernatant of SREBP-transfected cells (used for the results shown in bar c). FIG. 3B is a graph showing that increased expression of GOLPH2 reduces expression of IL-12-p35. The human IL-12 p35 promoter-luciferase reporter construct (see, Kim et al., Immunity 21, 643-53 (2004)) was used in RAW264.7 cells together with an effector construct that expressed human GOLPH2, or with a control empty vector (pCDNA3), at effector/reporter (E:R) molar ratios of 1:1, 2:1, and 4:1. Data are expressed as relative promoter activity, i.e. the ratio of IFN-γ/LPS-stimulated activity over unstimulated activity. FIG. 3C shows that GOLPH2 reduces expression from the IL-12p35 promoter but not from the IL-12 p40 promoter. HEK293 cells were transiently transfected with a FLAGged, empty expression vector (FLAG), or a FLAGged vector expressing human GOLPH2, or SREBP2. Forty-eight hours after transfection, cell-free culture supernatant was collected, and 0.5 ml was added to 1.5 ml of RAW264.7 cells transfected with human IL-12p35- or IL-12 p40-reporter construct for 6 h. RAW264.7 cells were then further stimulated with IFN-γ and LPS for 7 h before harvesting for luciferase activity measurement in triplicates. Data shown represent mean plus standard deviation. FIG. 3D shows that supernatants from apoptotic cells (AC) and from LPS-stimulated RAMOS cells reduce expression from the IL-12p35 promoter but not from the IL-12 p40 promoter. The same type of reporter assays described above for FIG. 3C were performed except that apoptotic cells (AC) or RAMOS culture supernatant were added to RAW264.7 cells.

FIG. 4A-B illustrates increased expression from the IL-12p35 promoter when GOLPH2 is inhibited by anti-GOLPH2 antibodies or by mutation of GOLPH2 at amino acid position 52 or 54. FIG. 4A illustrates expression from the IL-12p35 promoter in the absence and presence of anti-GOLPH2 antibodies. 2E2 supernatant (containing BDSF^{IL-12} activity) inhibited p35-promoter-driven transcription induced by IFN-γ and LPS in transfected RAW264.7 cells. Such expression was strongly and specifically inhibited by the addition of an anti-GOLPH2 polyclonal antibody (in amounts varying from 0-2 μg/ml). The anti-GOLPH2 polyclonal antibodies were GP73 (N-19) from Santa Cruz Biotechnologies (Santa Cruz, Calif.). Isotype-matched control IgG antibodies did not inhibit IL-12p35-promoter-driven transcription. FIG. 4B shows that mutant GOLPH2 does not inhibit IL-12p35-promoter-driven transcription. The IL-12 p35 reporter construct was cotransfected into RAW264.7 cells with control vector (pCR3.1), or wild type GOLPH2 (WT)-expression, secretion mutant R52A-expression, secretion-mutant R54A-expression, and Roquin-expression vectors in a molar ratio of effector to reporter (E:R) of 0.2:1. Luciferase activities were measured from cells following stimulation with IFN-γ and LPS. A low E:R ratio (1:0.2) was used to permit the interactive (synergistic) effects between GOLPH2 and Roquin to be optimally detected. When used at higher amounts, R52A and R54A were much less potent than the WT GOLPH2 (data not shown).

FIG. 5A-B illustrate identification of a BDSF^{IL-12}-responsive element within the IL-12p35 promoter. FIG. 5A shows different human IL-12p35 promoter sequences and expression levels from those promoters when they are tested in luciferase expression assays. Nucleic acid segments containing wild type and mutant IL-12p35 promoter sequences spanning nucleotide positions −1082 to +61 were separately linked to a nucleic acid encoding luciferase. The wild type IL-12p35 promoter segment (a) included a TGCCGCG sequence at nucleotide positions +13 to +19. A 3′ deletion of the IL-12p35 promoter segment (b) contained only the region spanning nucleotide positions −1082 to −4. Three mutant IL-12p35 promoter segments (c-e) had specific base-substitution mutations: XXCCGCG (c), TGXXGCG (d) and TGCCXXG (e). The promoter-reporter constructs were transfected into RAW264.7 cells, and co-cultured in the presence or absence of supernatant from 2E2 cells (containing BDSF^{IL-12}). Cells were stimulated with LPS for 7 h, and luciferase activity was measured from the cell lysates. As shown, the presence of BDSF^{IL-12} in the supernatant inhibits expression from the wild type IL-12p35 promoter, but such inhibition is lost when the promoter segment from nucleotide positions +13 to +19 is deleted (compare a vs. b), or mutated as in TGCCXXG (e) at positions +17 and +18 (compare a vs. e). FIG. 5B is a western blot showing that the presence of BDSF^{IL-12} in the supernatant of cultured cells leads to activation (phosphorylation) of GC-Binding Protein. RAW264.7 cells were cultured and exposed to medium (Med), or to apoptotic Jurkat cells (AC), or to supernatant from 2E2 cells (BDSF^{IL-12}) with or without IFNγ and LPS. Nuclear extracts were immunoprecipitated with anti-GC-Binding Protein antibodies (Kim et al., Immunity 21, 643-53 (2004)) followed by blotting with an anti-phospho-tyrosine mAb (pY99). Top panel: phosphorylated-GC-BP; bottom panel: total GC-BP (˜80 kDa). As shown, BDSF^{IL-12} stimulates tyrosine phosphorylation of GC-Binding Protein.

FIG. 6A-B illustrate B16 melanoma growth and immune responses in animals that do (wild type mice) and do not (IgM knockout B^{-/-} mice) express BDSF^{IL-12}. FIG. 6A shows B16 melanoma growth in WT and IgM knockout (B^{-/-}) mice. For tumor implantation, mice (five per group) were subcutaneously injected with 10⁶ tumor cells. Tumor growth was monitored periodically by measuring tumor diameters using a dial caliper. FIG. 6B shows expression levels of various T cell cytokines in wild type and IgM knockout B^{-/-} spleen and/or tumor cells. The spleens of tumor-inoculated mice (five per group) were collected. Splenocytes and tumors cells from these mice were cultured (8:1) for 7 days. Supernatants from these cultures were analyzed for cytokine levels by ELISAs. As shown, the cells from IgM knockout B^{-/-} mice exhibited heightened levels of expression of IL-10, INF-γ, p40 and IL-12.

FIG. 7 is a schematic diagram illustrating how GOLPH2 may induce inhibition of IL-12 production and T cell activation. IL-12 gene transcription is stimulated in professional antigen-presenting cells (dendritic cells (DCs) and macrophages) by innate immune cues, such as Toll-like receptor (TLR)-mediated signaling, and by adaptive immune signals such as CD40L (#1). These activate NF-κB and IL-12 p35 gene transcription (#3). Activated B lymphocytes (#4) and malignant B cells (#5) produce GOLPH2, which binds to a presumptive receptor (GOLPH2-R) on DCs (#6) and induces GC-BP tyrosine phosphorylation (#7). Phosphorylated GC-BP translocates to the nucleus (#8) and blocks IL-12 production by binding to the proximal p35 promoter region at the ACRE (Kim et al., Immunity 21, 643-53 (2004)) (#9). The lack of IL-12 (#10) results in a block of T_{H}1 differentiation and activation from naïve T (Th0) cells (#11), which limits cell-mediated immune responses against intracellular pathogens and malignant tumors. GC-BP phosphorylation is also induced in phagocytes that encounter apoptotic cells (ACs) with externalized phosphatidylserine (PS) through a phosphatidylserine receptor (#12).

### DETAILED DESCRIPTION

As described herein, Golgi phosphoprotein 2 (GOLPH2), initially dubbed BDSF^{IL-12} for B cell-derived soluble factor inhibiting IL-12, is a soluble factor produced by B cells, that surprisingly acts on dendritic cells and regulates T-cell-mediated immunity through the inhibition of IL-12, a potent activator of T_{H}1 cells. The regulation of T cell activity by GOLPH2 has significant clinical implications. Cytotoxic T-cells activated by _{TH}1 cytokines are a critical component of anti-viral and anti-tumor immunity. Viruses and tumor cells frequently use complex and elaborate strategies to escape immune attack during both initiation and invasion phases. The T_{H}1/T_{H}2 balance is impaired in many disorders, including HIV/AIDS, autoimmune diseases and malignancies. The role of B cells in regulating this delicate balance is largely underappreciated. As illustrated herein, BDSF^{IL-12}/GOLPH2 is produced by activated and malignant B cells, and provides a means for regulating and stimulating cellular immunity for anti-tumor, anti-viral and anti-microbial therapy.

In the following description, reference is made to various embodiments and the accompanying figures that form a part hereof, which are described and shown by way of illustration. These embodiments are described in detail to enable those skilled in the art to practice the invention, and it is to be understood that other embodiments may be utilized and that logical changes may be made without departing from the scope of the present invention. The following description of example embodiments is, therefore, not to be taken in a limited sense.

### Golgi Phosphoprotein 2

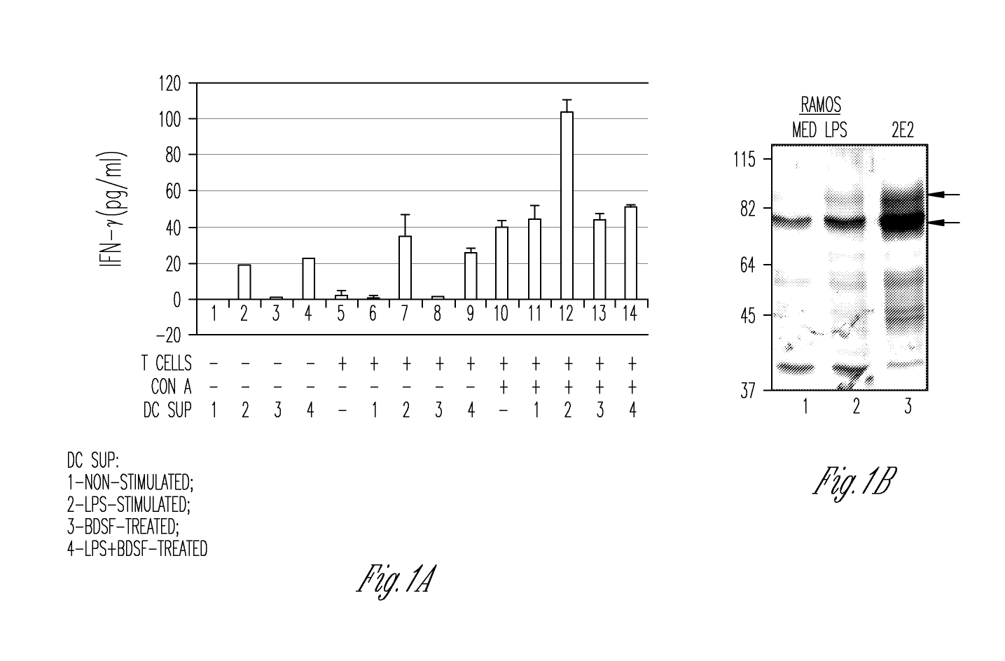

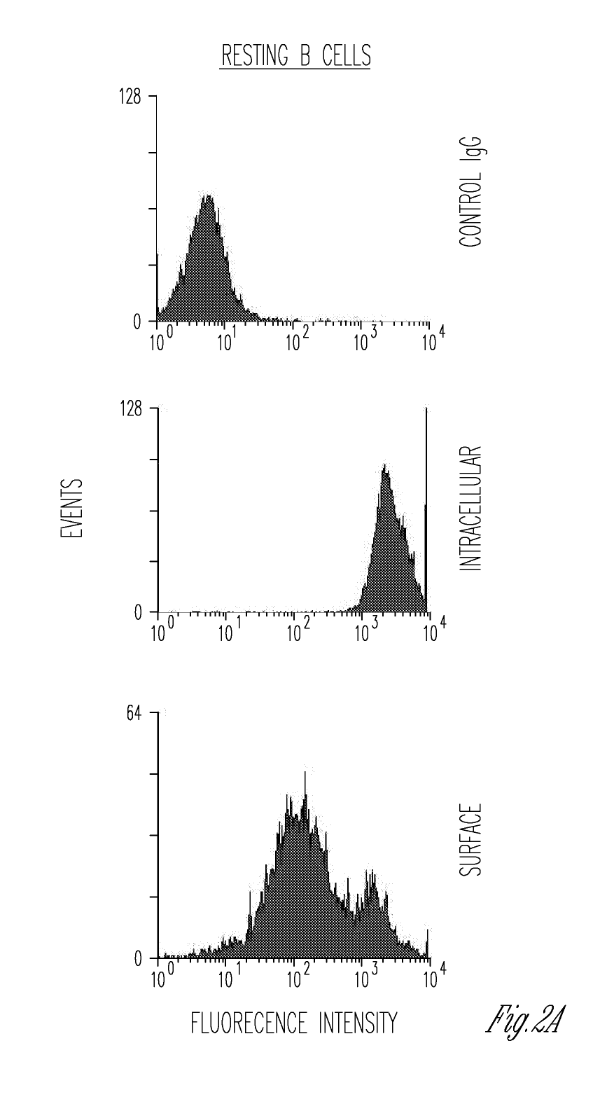

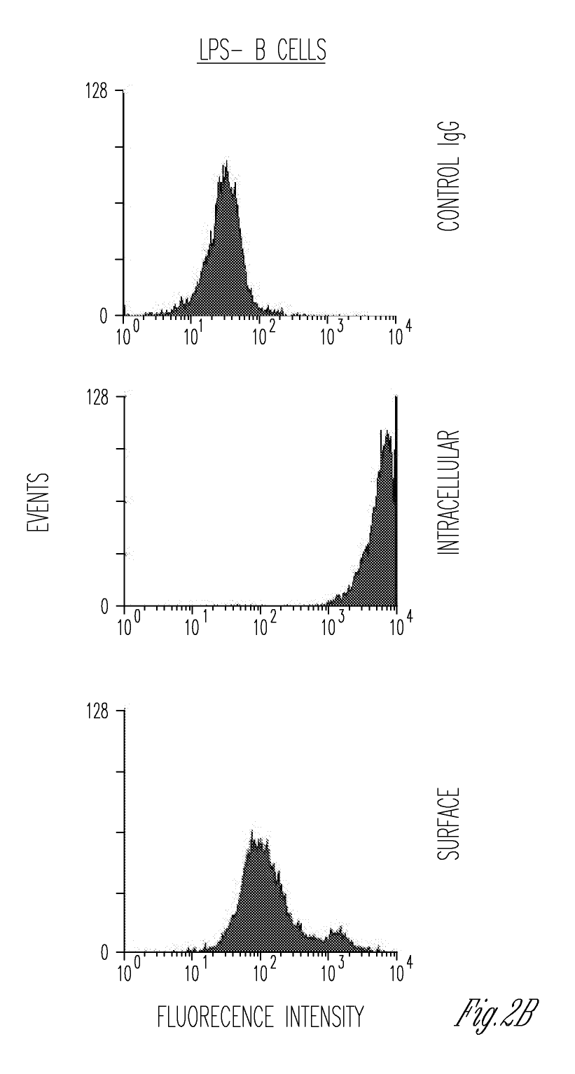

GOLPH2 is a Golgi phosphoprotein of previously unknown function. It is also called Golgi membrane protein1 (Golm1), GP73 and BDSF^{IL-12}. The inventors independently identified a soluble factor that was secreted by B cells and discovered that this factor inhibited IL-12 production by dendritic cells (FIG. 1A). This B-cell derived soluble factor was termed BDSF^{IL12}. Later experiments demonstrated that BDSF^{IL-12} was GOLPH2.

Experiments by the inventors identified several activities that are demonstrated by soluble, secreted BDSF^{IL-12}:
(i) BDSF^{IL-12} is highly resistant to trypsin and heat (e.g., boiling);
(ii) BDSF^{IL-12} selectively suppresses IL-12 secretion, but does not affect TNF-α, IL-10, IL-6 and TGF-β secretion;
(iii) BDSF^{IL-12} suppresses IL-12 secretion by activated monocytes and myeloid-derived dendritic cells in a manner independent of TGF-β, IL-10, TNF-α, and prostaglandin E2;
(iv) BDSF^{IL-12} has little effect on other dendritic cell properties such as surface expression of CD11c, CD80, CD86, and MHC II;
(v) BDSF^{IL-12}, in its soluble, extracellular form, activates a transcription factor to bind to the IL-12 p35 promoter—when bound the transcription factor inhibits transcription of IL-12 p35; and
(vi) Primary B cells co-cultured with HW-1-infected T cells produce BDSF^{IL-12} even though the B cells are not infected with HIV. These results indicate that the T_{H}1 impairment frequently observed in HIV-infected patients is caused, at least in part, by hyperactive B lymphocytes producing BDSF^{IL-12}.
   Additional properties of BDSF^{IL-12} (GOLPH2) are described throughout the application.

To further characterize BDSF^{IL12}, culture supernatants of LPS-stimulated RAMOS (B lymphoma) cells were treated with trypsin, boiled for 10 minutes, and the supernatants containing the soluble proteins were fractionated through an SDS-PAGE gel (FIG. 1B). The bands identified by the top arrow in lanes 1 and 2 in FIG. 1B were excised and analyzed by mass spectrometry. GOLPH2 along with several other proteins were identified as being present in the LPS-stimulated RAMOS supernatant, but absent in unstimulated RAMOS supernatant, which does not have the IL-12 inhibitory activity. Subsequent functional analyses ruled out other proteins except GOLPH2 as having the BDSF-like activity. Thus, the inventors determined that the BDSF^{IL12} factor was GOLPH2.

BDSF^{IL12}/GOLPH2 is widely expressed in normal epithelial cells of numerous tissues, especially in the gut, prostate, kidneys, lungs and within the central nervous system.

Under steady-state conditions, GOLPH2 is an integral membrane protein of the cis Golgi with an apparently benign function. However, as illustrated herein, it cycles out of the cis Golgi to endosomes and the cell surface to become a soluble factor that suppresses immune function. Endosomal trafficking of GOLPH2 allows for proprotein convertase furin-mediated cleavage, resulting in its release into the extracellular space. In its soluble form it is present in serum as a biomarker for human hepatocellular carcinoma (HCC).

The 73 kDa GOLPH2 protein is coded by the gene GOLM1 located on human chromosome 9q21.33 (mouse chromosome 13) and was originally cloned by differential screening of a cDNA library derived from liver tissue of a patient with adult giant-cell hepatitis (Kladney et al., Gene 249, 53-65 (2000).), a rare form of hepatitis with presumed viral etiology. GOLPH2 was independently identified in the secreted protein discovery initiative (SPDI), a large-scale effort to identify novel human secreted and transmembrane proteins using a biological signal sequence trap in yeast cells aided by computational tools.

The GOLPH2 gene is conserved in chimpanzee, dog, cow, mouse, chicken, and zebra fish. The closest human homologue to GOLPH2 is the cancer susceptibility candidate gene 4 (CASC4) protein (Swiss-Prot Q6P4E1), a single-pass type II membrane protein, the increased expression level of which is associated with HER-2/neu proto-oncogene overexpression.

Sequences for GOLPH2 are available for various GOLPH2 proteins and nucleic acids, for example, in the sequence database maintained by the National Center for Biotechnology Information (see website at www.ncbi.nlm.nih.gov/). The GOLPH2 protein, and segments or antigenic fragments thereof, are useful for generating inhibitors of GOLPH2 function. One example of a human GOLPH2 amino acid sequence is available as accession number CAG33482.1 (GI:48146519), provided below as SEQ ID NO:1.

```
    1 MGLGNGRRSM KSPPLVLAAL VACIIVLGFN YWIASSRSVD
  41 LQTRIMELEG RVRRAAAERG AVELKKNEFQ GELEKQREQL
  81 DKIQSSHNFQ LESVNKLYQD EKAVLVNNIT TGERLIRVLQ
121 DQLKTLQRNY GRLQQDVLQF QKNQTNLERK FSYDLSQCIN
161 QMKEVKEQCE ERIEEVTKKG NEAVASRDLS ENNDQRQQLQ
201 ALSEPQPRLQ AAGLPHTEVP QGKGNVLGNS KSQTPAPSSE
241 VVLDSKRRVE KEETNEIQVV NEEPQRDRLP QEPGREQVVE
281 DRPVGGRGFG GAGELGQTPQ VQAALSVSQE NPEMEGPERD
321 QLVIPDGQEE EQEAAGEGRN QQKLRGEDDY NMDENEAESE
361 TDKQAALAGN DRNIDVFNVE DQKRDTINLL DQREKRNHTL
```

This 400 amino acid GOLPH2 protein is cleaved between the two arginines after position 53 to generate a soluble form of GOLPH2 that can be secreted by the cell. The soluble form of the SEQ ID NO:1 GOLPH2 protein therefore has the following sequence (SEQ ID NO:2).

```
  54 RAAAERG AVELKKNEFQ GELEKQREQL
  81 DKIQSSHNFQ LESVNKLYQD EKAVLVNNIT TGERLIRVLQ
121 DQLKTLQRNY GRLQQDVLQF QKNQTNLERK FSYDLSQCIN
161 QMKEVKEQCE ERIEEVTKKG NEAVASRDLS ENNDQRQQLQ
201 ALSEPQPRLQ AAGLPHTEVP QGKGNVLGNS KSQTPAPSSE
241 VVLDSKRRVE KEETNEIQVV NEEPQRDRLP QEPGREQVVE
281 DRPVGGRGFG GAGELGQTPQ VQAALSVSQE NPEMEGPERD
321 QLVIPDGQEE EQEAAGEGRN QQKLRGEDDY NMDENEAESE
361 TDKQAALAGN DRNIDVFNVE DQKRDTINLL DQREKRNHTL
```

The GOLPH2 protein has a transmembrane region that includes a region spanning amino acid positions 12-34, and has the following amino acid sequence (SEQ ID NO:3): SPPLVLAALVACIIVLGFNYWIA. A GOLPH2 protein without the N-terminal region including such a transmembrane region has the following sequence (SEQ ID NO:4).

```
  35 SSRSVD
  41 LQTRIMELEG RVRRAAAERG AVELKKNEFQ GELEKQREQL
  81 DKIQSSHNFQ LESVNKLYQD EKAVLVNNIT TGERLIRVLQ
121 DQLKTLQRNY GRLQQDVLQF QKNQTNLERK FSYDLSQCIN
161 QMKEVKEQCE ERIEEVTKKG NEAVASRDLS ENNDQRQQLQ
201 ALSEPQPRLQ AAGLPHTEVP QGKGNVLGNS KSQTPAPSSE
241 VVLDSKRRVE KEETNEIQVV NEEPQRDRLP QEPGREQVVE
281 DRPVGGRGFG GAGELGQTPQ VQAALSVSQE NPEMEGPERD
321 QLVIPDGQEE EQEAAGEGRN QQKLRGEDDY NMDENEAESE
361 TDKQAALAGN DRNIDVFNVE DQKRDTINLL DQREKRNHTL
```

The GOLPH2 protein has a coiled-coil domain that includes a sequence spanning amino acid positions 35-203 of the SEQ ID NO:1 sequence. This sequence is shown below as SEQ ID NO:5.

```
  35 SSRSVD
  41 LQTRIMELEG RVRRAAAERG AVELKKNEFQ GELEKQREQL
  81 DKIQSSHNFQ LESVNKLYQD EKAVLVNNIT TGERLIRVLQ
121 DQLKTLQRNY GRLQQDVLQF QKNQTNLERK FSYDLSQCIN
161 QMKEVKEQCE ERIEEVTKKG NEAVASRDLS ENNDQRQQLQ
201 ALS
```

After cleavage and secretion, the GOLPH2 coiled-coil domain will be truncated at the N-terminus, and will have the following sequence (SEQ ID NO:6).

| | |
|---|---|
| 54 | RAAAERG AVELKKNEFQ GELEKQREQL |
| 81 | DKIQSSHNFQ LESVNKLYQD EKAVLVNNIT TGERLIRVLQ |
| 121 | DQLKTLQRNY GRLQQDVLQF QKNQTNLERK FSYDLSQCIN |
| 161 | QMKEVKEQCE ERIEEVTKKG NEAVASRDLS ENNDQRQQLQ |
| 201 | ALS |

These and other GOLPH2 protein segments may have utility for generating inhibitors of GOLPH2. For example, a GOLPH2 protein segment with amino acids 54-90, may have such utility. This GOLPH2 protein segment has the following sequence (SEQ ID NO:7).

| | |
|---|---|
| 54 | RAAAERG AVELKKNEFQ GELEKQREQL DKIQSSHNFQ |

Rabbit anti-GOLPH2 polyclonal antibodies (GP73 (N-19) that recognize the SEQ ID NO:7 GOLPH2 protein segment were effective inhibitors of GOLPH2.

Another GOLPH2 protein segment that may have utility for generating inhibitors of GOLPH2 includes, for example, a GOLPH2 protein segment with amino acids 91-130, having the following sequence (SEQ ID NO:8).

| | |
|---|---|
| 91 | LESVNKLYQD EKAVLVNNIT TGERLIRVLQ DQLKTLQRNY |

Another GOLPH2 protein segment that may have utility for generating inhibitors of GOLPH2 includes, for example, a GOLPH2 protein segment with amino acids 131-170, having the following sequence (SEQ ID NO:9).

| | |
|---|---|
| 131 | GRLQQDVLQF QKNQTNLERK FSYDLSQCIN QMKEVKEQCE |

Another GOLPH2 protein segment that may have utility for generating inhibitors of GOLPH2 includes, for example, a GOLPH2 protein segment with amino acids 171-210, having the following sequence (SEQ ID NO:10).

| | |
|---|---|
| 171 | ERIEEVTKKG NEAVASRDLS ENNDQRQQLQ ALSEPQPRLQ |

Another GOLPH2 protein segment that may have utility for generating inhibitors of GOLPH2 includes, for example, a GOLPH2 protein segment with amino acids 211-250, having the following sequence (SEQ ID NO:11).

| | |
|---|---|
| 211 | AAGLPHTEVP QGKGNVLGNS KSQTPAPSSE VVLDSKRRVE |

Another GOLPH2 protein segment that may have utility for generating inhibitors of GOLPH2 includes, for example, a GOLPH2 protein segment with amino acids 251-290, having the following sequence (SEQ ID NO:12).

| | |
|---|---|
| 251 | KEETNEIQVV NEEPQRDRLP QEPGREQVVE DRPVGGRGFG |

Another GOLPH2 protein segment that may have utility for generating inhibitors of GOLPH2 includes, for example, a GOLPH2 protein segment with amino acids 291-330, having the following sequence (SEQ ID NO:13).

| | |
|---|---|
| 291 | GAGELGQTPQ VQAALSVSQE NPEMEGPERD QLVIPDGQEE |

Another GOLPH2 protein segment that may have utility for generating inhibitors of GOLPH2 includes, for example, a GOLPH2 protein segment with amino acids 331-370, having the following sequence (SEQ ID NO:14).

| | |
|---|---|
| 331 | EQEAAGEGRN QQKLRGEDDY NMDENEAESE TDKQAALAGN |

Another GOLPH2 protein segment that may have utility for generating inhibitors of GOLPH2 includes, for example, a GOLPH2 protein segment with amino acids 371-400, having the following sequence (SEQ ID NO:15).

| | |
|---|---|
| 371 | DRNIDVFNVE DQKRDTINLL DQREKRNHTL |

A nucleic acid sequence that encodes the above GOLPH2 proteins (SEQ ID NOs: 1-15) is available as accession number CR457201.1 (GI:48146518) and provided below as nucleic acid SEQ ID NO:16.

| | |
|---|---|
| 1 | ATGGGCTTGG GAAACGGGCG TCGCAGCATG AAGTCGCCGC |
| 41 | CCCTCGTGCT GGCCGCCCTG GTGGCCTGCA TCATCGTCTT |
| 81 | GGGCTTCAAC TACTGGATTG CGAGCTCCCG GAGCGTGGAC |
| 121 | CTCCAGACAC GGATCATGGA GCTGGAAGGC AGGGTCCGCA |
| 161 | GGGCGGCTGC AGAGAGAGGC GCCGTGGAGC TGAAGAAGAA |
| 201 | CGAGTTCCAG GGAGAGCTGG AGAAGCAGCG GGAGCAGCTT |
| 241 | GACAAAATCC AGTCCAGCCA CAACTTCCAG CTGGAGAGCG |
| 281 | TCAACAAGCT GTACCAGGAC GAAAAGGCGG TTTTGGTGAA |
| 321 | TAACATCACC ACAGGTGAGA GGCTCATCCG AGTGCTGCAA |
| 361 | GACCAGTTAA AGACCCTGCA GAGGAATTAC GGCAGGCTGC |
| 401 | AGCAGGATGT CCTCCAGTTT CAGAAGAACC AGACCAACCT |
| 441 | GGAGAGGAAG TTCTCCTACG ACCTGAGCCA GTGCATCAAT |
| 481 | CAGATGAAGG AGGTGAAGGA ACAGTGTGAG GAGCGAATAG |
| 521 | AAGAGGTCAC CAAAAAGGGG AATGAAGCTG TAGCTTCCAG |
| 561 | AGACCTGAGT GAAAACAACG ACCAGAGACA GCAGCTCCAA |
| 601 | GCCCTCAGTG AGCCTCAGCC CAGGCTGCAG GCAGCAGGCC |
| 641 | TGCCACACAC AGAGGTGCCA CAAGGGAAGG GAAACGTGCT |
| 681 | TGGTAACAGC AAGTCCCAGA CACCAGCCCC CAGTTCCGAA |
| 721 | GTGGTTTTGG ATTCAAAGAG ACGAGTTGAG AAAGAGGAAA |
| 761 | CCAATGAGAT CCAGGTGGTG AATGAGGAGC CTCAGAGGGA |
| 801 | CAGGCTGCCG CAGGAGCCAG GCCGGGAGCA GGTGGTGGAA |
| 841 | GACAGACCTG TAGGTGGAAG AGGCTTCGGG GGAGCCGGAG |
| 881 | AACTGGGCCA GACCCCACAG GTGCAGGCTG CCCTGTCAGT |
| 921 | GAGCCAGGAA AATCCAGAGA TGGAGGGCCC TGAGCGAGAC |
| 961 | CAGCTTGTCA TCCCCGACGG ACAGGAGGAG GAGCAGGAAG |
| 1001 | CTGCCGGGGA AGGGAGAAAC CAGCAGAAAC TGAGAGGAGA |
| 1041 | AGATGACTAC AACATGGATG AAAATGAAGC AGAATCTGAG |
| 1081 | ACAGACAAGC AAGCAGCCCT GGCAGGGAAT GACAGAAACA |
| 1121 | TAGATGTTTT TAATGTTGAA GATCAGAAAA GAGACACCAT |
| 1161 | AAATTTACTT GATCAGCGTG AAAAGCGGAA TCATACACTT |
| 1201 | TAA |

Another example of a human GOLPH2 amino acid sequence is available as accession number CAG33482.1 (GI:48146519), provided below as SEQ ID NO:17.

| | |
|---|---|
| 1 | MMGLGNGRRS MKSPPLVLAA LVACIIVLGF NYWIASSRSV |
| 41 | DLQTRIMELE GRVRRAAAER GAVELKKNEF QGELEKQREQ |
| 81 | LDKIQSSHNF QLESVNKLYQ DEKAVLVNNI TTGERLIRVL |
| 121 | QDQLKTLQRN YGRLQQDVLQ FQKNQTNLER KFSYDLSQCI |
| 161 | NQMKEVKEQC EERIEEVTKK GNEAVASRDL SENNDQRQQL |
| 201 | QALSEPQPRL QAAGLPHTEV PQGKGNVLGN SKSQTPAPSS |
| 241 | EVVLDSKRQV EKEETNEIQV VNEEPQRDRL PQEPGREQVV |
| 281 | EDRPVGGRGF GGAGELGQTP QVQAALSVSQ ENPEMEGPER |
| 301 | DQLVIPDGQE EEQEAAGEGR NQQKLRGEDD YNMDENEAES |
| 361 | ETDKQAALAG NDRNIDVFNV EDQKRDTINL LDQREKRNHT |
| 401 | L |

This 401 amino acid GOLPH2 protein is cleaved between the two arginines after position 54, to give rise to the same soluble GOLPH2 protein with sequence SEQ ID NO:2.

A nucleic acid sequence for the above GOLPH2 SEQ ID NO:17 sequence is available as accession number AY358593.1 (GI:37182307) and provided below as nucleic acid SEQ ID NO:18.

| | |
|---|---|
| 1 | GCTCGAGGCC GGCGGCGGCG GGAGAGCGAC CCGGGCGGCC |
| 41 | TCGTAGCGGG GCCCCGGATC CCCGAGTGGC GGCCGGAGCC |
| 81 | TCGAAAAGAG ATTCTCAGCG CTGATTTTGA GATGATGGGC |
| 121 | TTGGGAAACG GGCGTCGCAG CATGAAGTCG CCGCCCCTCG |
| 161 | TGCTGGCCGC CCTGGTGGCC TGCATCATCG TCTTGGGCTT |
| 201 | CAACTACTGG ATTGCGAGCT CCCGGAGCGT GGACCTCCAG |
| 241 | ACACGGATCA TGGAGCTGGA AGGCAGGGTC CGCAGGGCGG |
| 281 | CTGCAGAGAG AGGCGCCGTG GAGCTGAAGA AGAACGAGTT |
| 321 | CCAGGGAGAG CTGGAGAAGC AGCGGGAGCA GCTTGACAAA |
| 361 | ATCCAGTCCA GCCACAACTT CCAGCTGGAG AGCGTCAACA |
| 401 | AGCTGTACCA GGACGAAAAG GCGGTTTTGG TGAATAACAT |
| 441 | CACCACAGGT GAGAGGCTCA TCCGAGTGCT GCAAGACCAG |
| 481 | TTAAAGACCC TGCAGAGGAA TTACGGCAGG CTGCAGCAGG |
| 521 | ATGTCCTCCA GTTTCAGAAG AACCAGACCA ACCTGGAGAG |
| 561 | GAAGTTCTCC TACGACCTGA GCCAGTGCAT CAATCAGATG |
| 601 | AAGGAGGTGA AGGAACAGTG TGAGGAGCGA ATAGAAGAGG |
| 641 | TCACCAAAAA GGGGAATGAA GCTGTAGCTT CCAGAGACCT |
| 681 | GAGTGAAAAC AACGACCAGA GACAGCAGCT CCAAGCCCTC |
| 721 | AGTGAGCCTC AGCCCAGGCT GCAGGCAGCA GGCCTGCCAC |
| 761 | ACACAGAGGT GCCACAAGGG AAGGGAAACG TGCTTGGTAA |
| 801 | CAGCAAGTCC CAGACACCAG CCCCCAGTTC CGAAGTGGTT |
| 841 | TTGGATTCAA AGAGACAAGT TGAGAAAGAG GAAACCAATG |
| 881 | AGATCCAGGT GGTGAATGAG GAGCCTCAGA GGGACAGGCT |
| 921 | GCCGCAGGAG CCAGGCCGGG AGCAGGTGGT GGAAGACAGA |
| 961 | CCTGTAGGTG GAAGAGGCTT CGGGGGAGCC GGAGAACTGG |
| 1001 | GCCAGACCCC ACAGGTGCAG GCTGCCCTGT CAGTGAGCCA |
| 1041 | GGAAAATCCA GAGATGGAGG GCCCTGAGCG AGACCAGCTT |
| 1081 | GTCATCCCCG ACGGACAGGA GGAGGAGCAG GAAGCTGCCG |
| 1121 | GGGAAGGGAG AAACCAGCAG AAACTGAGAG GAGAAGATGA |
| 1161 | CTACAACATG GATGAAAATG AAGCAGAATC TGAGACAGAC |
| 1201 | AAGCAAGCAG CCCTGGCAGG GAATGACAGA AACATAGATG |
| 1241 | TTTTTAATGT TGAAGATCAG AAAAGAGACA CCATAAATTT |
| 1281 | ACTTGATCAG CGTGAAAAGC GGAATCATAC ACTCTGAATT |
| 1321 | GAACTGGAAT CACATATTTC ACAACAGGGC CGAAGAGATG |
| 1361 | ACTATAAAAT GTTCATGAGG GACTGAATAC TGAAAACTGT |
| 1401 | GAAATGTACT AAATAAAATG TACATCTGA |

Structural analysis has revealed that GOLPH2 is entirely helical after the transmembrane region, with two predicted continuous helical regions of 150 to 200 residues in length. This striking helical nature may explain its resistance to proteases, because proteolysis requires a stretch of extended structure such as β-strand or random coil conformation. The apparent simplicity in the secondary structure of GOLPH2 may also explain its heat resistance because the protein may have an extraordinarily high denaturation temperature or may re-fold readily upon cooling.

Studies have identified high levels of GOLPH2 in the sera of patients with liver disease, particularly hepatocellular carcinoma (HCC) (Li & Fan, Hepatology 50, 1682 (2009); Marrero et al., J Hepatol 43, 1007-12 (2005)). Compared with α-fetoprotein, the most commonly used serum marker for carcinoma, GOLPH2 serum levels appear to be more sensitive for early HCC (Marrero et al., J Hepatol 43, 1007-12 (2005)). GOLPH2 is hyperfucosylated in HCC, and its hyperfucosylated fraction in serum is an even better disease marker (Block et al., Proc Natl Acad Sci USA 102, 779-84 (2005)). The most profound elevation of serum levels of GOLPH2 are detected in patients who had developed HCC on the background of HCV genotype 1b infection (Riener et al., Hepatology 49, 1602-9 (2009)). The level of serum GOLPH2 is also significantly elevated in lung cancer patients (Zhang et al., Clin Biochem 43, 983-91 (2010)). GOLPH2 is also described as an excellent ancillary tissue biomarker for the diagnosis of prostate cancer (Kristiansen et al., Br. J. Cancer 99: 939-48 (2008)).

Transgenic mice expressing a C-terminally truncated GOLPH2 exhibit decreased survival and hepato-renal pathology with strong inflammatory cell infiltrates (Wright et al., Int J Clin Exp Pathol 2, 34-47 (2009)). This renal pathology is somewhat similar to that observed in mice with a knockout for the lipoprotein clusterin (CLU) (Whelchel et al., Invest Ophthalmol V is Sci 34, 2603-6 (1993)), the secretory form of which (sCLU) has been shown to interact with secretory GOLPH2 through the latter's C-terminus (Li & Fan, Hepatology 50, 1682 (2009)).

As described herein, GOLPH2 has a heretofore unknown and unexpected function: regulating IL-12 production by dendritic cells and IL-12-driven T_{H}1 activation. Experiments described herein demonstrate the cellular and molecular mechanisms of GOLPH2 and its impact on cell-mediated resistance to tumor growth and immune escape. As further demonstrated herein, compositions and methods for inhibiting GOLPH2 increase IL-12 expression and reduce the immunosuppressive activity that GOLPH2 normally exhibits.

One of the traditional immunological paradigms is that B-cell and T-cell interactions are a one-way phenomenon of T-cell help to induce the terminal differentiation of B cells to immunoglobulin class-switched plasma cells. Studies described herein challenge this dogma, and define a specific molecule in this missing link: GOLPH2, which as illustrated herein is a novel target for cancer therapy.

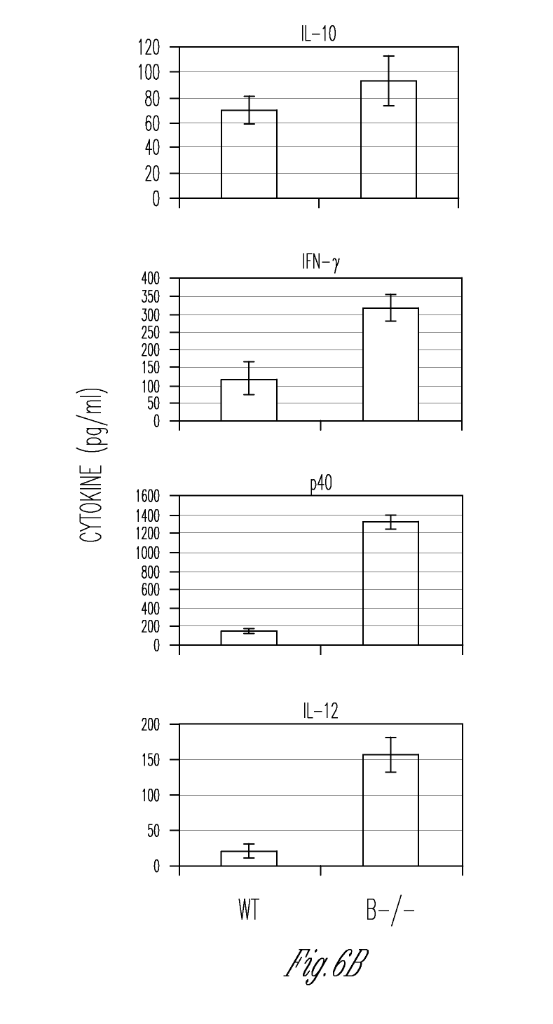

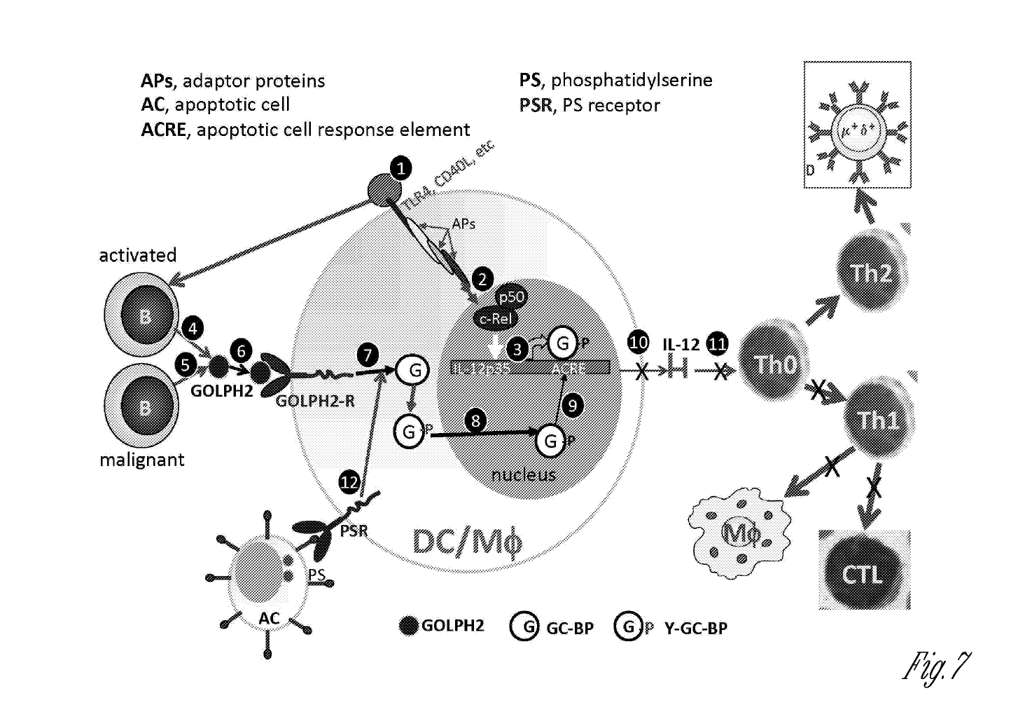

FIG. 7 depicts the proposed model of GOLPH2-induced inhibition of IL-12 production and T cell activation. IL-12 gene transcription is stimulated in professional antigen-presenting cells (DCs and macrophages) by innate immune cues, such as TLR-mediated signaling, and by adaptive immune signals such as CD40L (#1). These activate NF-κB and IL-12 p35 gene transcription (#3). Activated B lymphocytes (#4) and malignant B cells (#5) produce GOLPH2, which binds to a presumptive receptor (GOLPH2-R) on DCs (#6) and induces GC-BP tyrosine phosphorylation (#7). Phosphorylated GC-BP translocates to the nucleus (#8) and blocks IL-12 production by binding to the proximal p35 promoter region at the ACRE (Kim et al., Immunity 21, 643-53 (2004)) (#9). The lack of IL-12 (#10) results in a block of T_{H}1 differentiation and activation from naïve T (Th0) cells (#11), which limits cell-mediated immune responses against intracellular pathogens and malignant tumors. GC-BP phosphorylation is also induced in phagocytes that encounter apoptotic cells (ACs) with externalized phosphatidylserine (PS) through a phosphatidylserine receptor (#12).

### Methods of Treatment

One aspect of the invention is a method of enhancing cell-mediated immunity in a mammal in need thereof that includes administering to the mammal an inhibitor of GOLPH2 to thereby enhance cell-mediated immunity in the mammal. Cell-mediated immunity is an immune response that does not involve antibodies but rather involves the activation of macrophages, natural killer cells (NK), antigen-specific cytotoxic T-lymphocytes, and the release of various cytokines in response to an antigen.

As illustrated herein, inhibitors of GOLPH2 increase the mammal's endogenous production of IL-12. In some embodiments, the inhibitors of GOLPH2 increase the mammal's endogenous production of IL-12 by 10%, or 20%, or 50%, or 70%, or 100%, or 150%, or 200%, or 300%, or 400%, or 500%, or %700, or 1000%.

Inhibitors of GOLPH2 can also increase interferon-γ (IFN-γ) production by activated T lymphocytes. In some embodiments, the inhibitors of GOLPH2 increase the mammal's endogenous production of T lymphocyte IFN-γ by 10%, or 20%, or 50%, or 70%, or 100%, or 150%, or 200%, or 300%, or 400%, or 500%, or %700, or 1000%.

The methods and compositions described herein can be used to treat a variety of cancers and tumors, for example, leukemia, sarcoma, osteosarcoma, lymphomas, melanoma, glioma, pheochromocytoma, hepatoma, ovarian cancer, skin cancer, testicular cancer, gastric cancer, pancreatic cancer, renal cancer, breast cancer, prostate cancer, colorectal cancer, cancer of head and neck, brain cancer, esophageal cancer, bladder cancer, adrenal cortical cancer, lung cancer, bronchus cancer, endometrial cancer, nasopharyngeal cancer, cervical or liver cancer, and cancer at an unknown primary site.

Examples of liver diseases that can be treated include those involving hepatitis viruses and liver disorders associated with acute or chronic viral hepatitis (such as hepatitis B and hepatitis C), or cirrhosis or hepatocellular carcinoma caused by hepatitis C. Hepatitis B is defined as hepatitis caused by HBV infection, and Hepatitis C is defined as hepatitis caused by HCV infection. Chronic hepatitis is defined as a clinical condition where inflammation in the liver persists, or appears to persist, for 6 months or more. Liver disorders are defined as inflammatory diseases in the liver, and may be used as a concept including fatty liver, cirrhosis, and hepatocellular carcinoma according to the progression of symptoms.

The methods and compositions described herein can also be used to treat a variety of microbial infections involving, for example, bacteria, yeasts, viruses, viroids, molds, fungi, and other microorganisms.

For example, the infection to be treated may be resulted to infection by a pathogenic bacteria, such as Shigella species, Salmonella typhi, Salmonella typhimurium, Yersinia enterocolitica, Yersinia pestis, Vibrio cholerae, Campylobacter jejuni, Helicobacter jejuni, Pseudomonas aeruginosa, Haemophilus influenzae, Bordetella pertussis (whooping cough), Vibrio cholerae, and E. coli, including Diarrheagenic E. Coli, enteroaggregative E. coli (EaggEC), enterohaemorrhagic E. coli (EHEC), enteroinvasive E. coli (EIEC), enteropathogenic E. coli (EPEC) and enterotoxigenic E. coli (ETEC), Uropathogenic E. coli (UPEC), and neonatal meningitis E. coli (NMEC). Other pathogenic bacterial infections that may be treated include infections by Bacilus anthracis, Clostridium botulinum, Francisella tularensis, Burkholderia pseudomallei, Coxiella bumetti, Brucella species, Burkholderia mallei, Staphylococcus, drug-resistant Streptococcus, Rickettsia prowazekii, Shigella species, Salmonella, Listeria monocytogenes, Camp ylobacterjeluni, and Yersinia enterocolitica.

A variety of viral infections can be treated or prevented by the compositions described herein, including, but not limited to, Hepatitis A, Hepatitis B, Hepatitis C, Human Immunodeficiency Virus, Respiratory Syncytial Virus, Cytomegalo Virus, Herpes Simplex Virus, Ectocarpus Siliculosus Virus, Vesicular Stomatital Virus, viral encephalitides (such as Eastern equine encephalomyelitis virus, Venezuelan equine encephalomyelitis virus, and Western equine encephalomyelitis virus), viral hemorrhagic fevers (such as Ebola, Marburg, Junin, Argentine, and Lassa), influenza viruses, and avian influenza viruses (sometimes called bird flu). Other viral infections that may be treated include, but not limited to those involving Variola major (smallpox) and other pox viruses, Arenaviruses (including LCM, Junin viruses, Machupo viruses, Guanarito viruses, Lassa Fever viruses), Bunyaviruses (including Hantaviruses, Rift Valley Fever viruses), Flaviruses (including Dengue viruses), Filoviruses (including Ebola viruses and Marburg viruses), Tickbome hemorrhagic fever viruses (including Crimean-Congo Hemorrhagic fever viruses), Tickbome encephalitis viruses, yellow-feverviruses, influenza viruses, Rabies virus, West Nile Viruses, La Crosse viruses, California encephalitis viruses, Venezuelan Equine Encephalomyelitis viruses, Eastern Equine Encephalomyelitis viruses, Western Equine Encephalomyelitis viruses, Japanese Encephalitis Viruses, and Kyasanur Forest Viruses.

### The Anti-Tumor Role of IL-12

IL-12 can dramatically activate the host's immune apparatus against a variety of tumors in animal models. The anti-tumor efficacy of IL-12 is mediated via the activation of natural killer (NK) cells for non-antigen specific, MHC I-dictated cytotoxicity, as well as induction of T_{H}1 effector cells and activation of cytotoxic T lymphocyte (CTL) for tumor-specific elimination and long-term protective immunity. The ability of IL-12 to activate five important immune effector cells [NK, CTL, T helper (T_{H}), lymphoid tissue-inducer (LTi) cells, dendritic cells (DCs) and macrophages] leaves tumors little chance to escape. As described herein, signaling does occur from B-cells that modulates the differentiation of T-cells, including T_{H}1 differentiation.

The lack of apparent immunogenicity of many tumors in situ may be due to special properties of the tumor cells, for example, a lack of costimulatory molecules, down-regulation of MHC molecules, or production of immunosuppressive factors. Such lack of immunogenicity may also be due to intrinsic tolerance mechanisms of the immune system. IL-12 is able to dramatically overcome the poor anti-tumor immune response and provide tumor-specific elimination and long-term protective immunity.

IL-12 activates five important immune effector cells: natural killer cells, cytotoxic T lymphocytes, T helper (T_{H}) cells, lymphoid tissue-inducer (LTi) cells, dendritic cells (DCs) and macrophages. The combined action of these IL-12-activated cells leaves tumors with little chance to escape a host's immune system. Thus, if IL-12 production is enhanced the lack of immunogenicity of tumor cells can be overcome, and the host's own immune system can eliminate cancer cells without the need for debilitating chemotherapy.

Initial results from human clinical applications of IL-12 for human T cell lymphoma, B cell non-Hodgkin lymphoma, melanoma, and renal carcinoma, and SW-infection model in rhesus macaques support the potential of IL-12 as an anti-tumor therapeutic. See, Rook et al. Blood 94, 902-8. (1999); Rook et al. Ann N Y Acad Sci 941, 177-84. (2001); Ansell et al. Blood 99, 67-74 (2002); Mortarini et al. Cancer Res 60, 3559-68. (2000); Gollob et al. Clin Cancer Res 6, 1678-92. (2000); Lee et al. J Clin Oncol 19, 3836-47. (2001); Kang et al. Hum Gene Ther 12, 671-84. (2001); Gajewski et al., Clin Cancer Res 7, 895s-901s. (2001); Portielje et al. Clin Cancer Res 5, 3983-9. (1999); Ansari et al. J Virol 76, 1731-43. (2002).

Following a brief period of uncertainty about the safety of recombinant IL-12 and intense investigations into the causes of its undesirable effects, there is a recent resurgence in its use in more rationally designed cancer treatment, such as combination therapy and vaccine adjuvant, for example, for peritoneal carcinoma associated with ovarian cancer or primary peritoneal carcinoma (Lenzi et al. J Transl Med 5, 66 (2007)), AIDS-related Kaposi sarcoma (Little et al., Blood 110, 4165-71 (2007)), relapsed refractory non-Hodgkin's lymphoma and Hodgkin's disease (Younes et al., Clin Cancer Res 10, 5432-8 (2004)), and advanced melanoma (Peterson et al. J Clin Oncol 21, 2342-8 (2003)).

### T_{H}1/T_{H}2 Imbalance in Malignancies

Increasing clinical and experimental evidence indicates that early and persistent inflammatory-type responses in or around developing neoplasms regulate many aspects of tumor development (de Visser et al., Nat Rev Cancer 6, 24-37 (2006)). It is now appreciated that persistent humoral immune responses exacerbate recruitment and activation of innate immune cells in neoplastic microenvironments where they regulate tissue remodeling, pro-angiogenic and pro-survival pathways that together potentiate cancer development (Andreu et al., Cancer Cell 17, 121-134 (2010)). Pre-malignant and malignant tissues are known to be associated with alterations in immune cell functions, including suppressed cell-mediated immunity (CMI), associated with failure to reject tumors, in combination with enhanced humoral immunity that can potentiate tumor promotion and progression (Dalgleish et al., Adv Cancer Res 84, 231-76 (2002)). Numerous human and animal model studies have demonstrated that T_{H}1 and T_{H}2 cytokine balances critically affect the progression of various cancers (Agarwal et al. Cancer Immunol Immunother 55, 734-43 (2006); Kanazawa et al. Anticancer Res 25, 443-9 (2005); Galon et al. Science 313, 1960-4 (2006); Sheu et al. J Immunol 167, 2972-8 (2001)).

The T_{H}1/T_{H}2 imbalance may reflect significant changes in cellular immunity, in well documented cases of hematological malignancies, in children and adults with acute lymphoblastic leukemia (ALL), in chronic lymphocytic leukemia (CLL), in colorectal adenoma-carcinoma, and during ovarian cancer progression. See, Mori et al. Cancer Immunol Immunother 50, 566-8 (2001); Zhang et al. Cancer Immunol Immunother 49, 165-72 (2000); Yotnda et al. Exp Hematol 27, 1375-83 (1999); de Totero et al. Br J Haematol 104, 589-99 (1999); Cui et al. Cancer Immunol Immunother 56, 1993-2001 (2007); Kusuda et al. Oncol Rep 13, 1153-8 (2005).

### B Cell Regulation of T Cell Responses Via Dendritic Cells

One of the traditional immunological dogmas is that B-cell and T-cell interactions are a one-way phenomenon of T-cell help to induce the terminal differentiation of B cells to immunoglobulin class-switched plasma cells. However, recent studies indicate that B cells have a reciprocal influence on T-cell differentiation and effector function. For example, B cells can induce direct tolerance of antigen specific CD8⁺ T cells, induce T-cell anergy via transforming growth factor-beta (TGF-β) production, down-regulate IL-12 production by dendritic cells, and influence T_{H}1/T_{H}2 differentiation via the production of regulatory cytokines (Bennett et al., J Exp Med 188, 1977-83 (1998); Eynon & Parker, J Exp Med 175, 131-8 (1992); Fuchs et al., Science 258, 1156-9 (1992); Parekh et al., J Immunol 170, 5897-911 (2003); Skok et al., J Immunol 163, 4284-91 (1999); Mori et al., J Exp Med 176, 381-8 (1992); Harris et al., Nat Immunol 1, 475-82 (2000)). Similarly, B cells can exert a regulatory function within in vivo models of T-cell immunity including tumor rejection, experimental autoimmune encephalitis (EAE), and rheumatoid arthritis (RA) (Qin et al., Nat Med 4, 627-30 (1998); Fillatreau et al., Nat Immunol 3, 944-50 (2002); Mauri et al., J Exp Med 197, 489-501 (2003)). In mice, a relatively rare spleen B cell subset with IL-10-dependent negative T-cell-regulating function has recently been identified and named B10 cells (Matsushita et al. J Clin Invest 118, 3420-30 (2008); Watanabe et al., J Immunol 184, 4801-9 (2010); Yanaba et al., Immunity 28, 639-50 (2008)). It was shown in the experimental autoimmune encephalomyelitis (EAE) model that B 10 cells indirectly modulate the T cell-mediated autoimmunity by inhibiting the ability of dendritic cells to act as antigen-presenting cells (APCs)(Matsushita et al. J Immunol 185, 2240-52 (2010)). B cells can inhibit the ability of dendritic cells vaccination to provide protection from tumor growth (Watt et al. J Immunother 30, 323-32 (2007)). Inhibition of dendritic cell induced immunity by B cells was independent of presentation of major histocompatibility molecule (MHC) class-I bound tumor antigen but dependent on B-cell expression of MHC class-II. Administration of B cells did not alter the ability of dendritic cells to migrate from the injection site or impair dendritic cell-T cell interactions within the draining lymph node. The inhibitory effect of B cells was partially reversed by the depletion of CD4⁺, CD25⁺ regulatory T cells (Watt et al., J Immunother 30, 323-32 (2007)). Thus, B cells represent an important but so far underappreciated regulator of T cell-mediated immunity.

### Antibodies Against GOLPH2

The invention also provides antibodies and binding entities that preferentially bind to GOLPH2 protein. The anti-GOLPH2 antibodies and binding entities of the invention can bind to any epitope on the GOLPH2 protein. For example, the anti-GOLPH2 antibodies and binding entities can bind to any epitope within GOLPH2 polypeptides having any of SEQ ID NO: 1-15, and 17. However, the anti-GOLPH2 antibodies and binding entities preferably bind with specificity to GOLPH2 in its soluble, extracellular form. Examples of GOLPH2 polypeptide sequences to which the anti-GOLPH2 antibodies/binding entities can bind include GOLPH2 polypeptides with any of SEQ ID NO:2, 4-15.

The GOLPH2 epitopes to which the anti-GOLPH2 antibodies and/or binding entities can bind can include any GOLPH2 peptide sequence with a segment length, for example, of about 10-20 amino acids. Thus, GOLPH2 epitopes can be employed for generating anti-GOLPH2 antibodies and/or binding entities from polypeptides having any of SEQ ID NO: 1-15, and 17 or any analog thereof. Thus, in some embodiment, the GOLPH2 epitope can be a truncated polypeptide, for example, any of SEQ ID NO: 1-15, and 17 with any number of amino acids removed from the N-terminal and/or C-terminal end. For example, truncated SEQ ID NO: 1-15, and 17 polypeptides with 1, 2, 3, 4, 5, 6, 7, 8, 9, 10, 11, 12, 13, 14, 15, 16, 17, 18, 19, 20, 21, 22, 23, 24, 25, 30, 40, 50 or 60 amino acid(s) deleted from the N-terminal and/or C-terminal end can be used as epitopes for generating anti-GOLPH2 antibodies and/or binding entities. In other embodiments, the GOLPH2 epitope can be a polypeptide with one or more amino acid substitutions. For example, the GOLPH2 epitope can be a polypeptide with any of the SEQ ID NO: 1-15, and 17 sequences where 1, 2, 3, 4, 5, 6, 7, 8, 9, 10, 11, 12, 13, 14, 15, 16, 17, 18, 19, 20, 21, 22, 23, 24, 25, 30, 40, 50 or 60 amino acid(s) are replaced with another amino acid. In some embodiments, the substituted amino acid(s) have a similar chemical structure or similar chemical properties.

Anti-GOLPH2 antibodies and/or binding entities that specifically bind to such GOLPH2 epitopes are useful for inhibiting the function of secreted GOLPH2. As described herein, when GOLPH2 is cleaved and secreted, it inhibits the immune response, for example, by inhibiting production of IL-12. However, administration of inhibitors of secreted GOLPH2 can reduce the inhibition and stimulate an immune response.

The invention therefore provides antibodies and binding entities made by available procedures that can bind GOLPH2, especially soluble, secreted GOLPH2. Antibodies that inhibit GOLPH2 function and restore expression of IL-12 are preferred. For therapeutic purposes, human or humanized anti-GOLPH2 antibodies are preferred. Thus, the binding domains of antibodies or binding entities, for example, the CDR regions of antibodies with specificity for GOLPH2, can be transferred into or utilized with any convenient binding entity backbone, including a human antibody backbone.

Antibody molecules belong to a family of plasma proteins called immunoglobulins whose basic building block, the immunoglobulin fold or domain, is used in various forms in many molecules of the immune system and other biological recognition systems. A typical antibody is a tetrameric structure consisting of two identical immunoglobulin heavy chains and two identical light chains and has a molecular weight of about 150,000 daltons.

The heavy and light chains of an antibody consist of different domains.

Each light chain has one variable domain (VL) and one constant domain (CL), while each heavy chain has one variable domain (VH) and three or four constant domains (CH). See, e.g., Alzari, P. N., Lascombe, M.-B. & Poljak, R. J. (1988) Three-dimensional structure of antibodies. Annu. Rev. Immunol. 6, 555-580. Each domain, consisting of about 110 amino acid residues, is folded into a characteristic β-sandwich structure formed from two β-sheets packed against each other, the immunoglobulin fold. The VH and VL domains each have three complementarity determining regions (CDR1-3) that are loops, or turns, connecting β-strands at one end of the domains. The variable regions of both the light and heavy chains generally contribute to antigen specificity, although the contribution of the individual chains to specificity is not always equal. Antibody molecules have evolved to bind to a large number of molecules by using six randomized loops (CDRs).

Immunoglobulins can be assigned to different classes depending on the amino acid sequences of the constant domain of their heavy chains. There are at least five (5) major classes of immunoglobulins: IgA, IgD, IgE, IgG and IgM. Several of these may be further divided into subclasses (isotypes), for example, IgG-1, IgG-2, IgG-3 and IgG-4; IgA-1 and IgA-2. The heavy chain constant domains that correspond to the IgA, IgD, IgE, IgG and IgM classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of variable domains differ extensively in sequence from one antibody to the next. The variable domains are for binding and determine the specificity of each particular antibody for its particular antigen. However, the variability is not evenly distributed through the variable domains of antibodies. Instead, the variability is concentrated in three segments called complementarity determining regions (CDRs), also known as hypervariable regions in both the light chain and the heavy chain variable domains.

The more highly conserved portions of variable domains are called framework (FR) regions. The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from another chain, contribute to the formation of the antigen-binding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody which includes the variable domain complementarity determining regions (CDR), and the like forms, all of which fall under the broad term “antibody,” as used herein. The present invention contemplates the use of any specificity of an antibody, polyclonal or monoclonal, and is not limited to antibodies that recognize and immunoreact with a specific GOLPH2 polypeptide or derivative thereof.

Moreover, the binding regions, or CDR, of antibodies can be placed within the backbone of any convenient binding entity polypeptide. In preferred embodiments, in the context of methods described herein, an antibody, binding entity or fragment thereof that is not immunogenic to a mammal to be treated is used. Also preferred are antibodies, binding entities or fragments thereof that are immunospecific for GOLPH2, as well as the variants and derivatives thereof.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called Fab fragments, each with a single antigen binding site, and a residual Fc fragment. Fab fragments thus have an intact light chain and a portion of one heavy chain. Pepsin treatment yields an F(ab′)₂ fragment that has two antigen binding fragments that are capable of cross-linking antigen, and a residual fragment that is termed a pFc' fragment. Fab′ fragments are obtained after reduction of a pepsin digested antibody, and consist of an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site. As used herein, “functional fragment” with respect to antibodies, refers to Fv, F(ab) and F(ab′)₂ fragments.

Additional fragments can include diabodies, linear antibodies, single-chain antibody molecules, and multispecific antibodies formed from antibody fragments. Single chain antibodies are genetically engineered molecules containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N. Y., pp. 269-315 (1994).

The term “diabodies” refers to a small antibody fragments with two antigen-binding sites, where the fragments comprise a heavy chain variable domain (VH) connected to a light chain variable domain (VL) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad. Sci. USA 90: 6444-6448 (1993).

Antibody fragments contemplated by the invention are therefore not full-length antibodies. However, such antibody fragments can have similar or improved immunological properties relative to a full-length antibody. Such antibody fragments may be as small as about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more.

In general, an antibody fragment or binding entity of the invention can have any upper size limit so long as it is has similar or improved immunological properties relative to an antibody that binds with specificity to a GOLPH2 polypeptide. For example, smaller binding entities and light chain antibody fragments can have less than about 200 amino acids, less than about 175 amino acids, less than about 150 amino acids, or less than about 120 amino acids if the antibody fragment is related to a light chain antibody subunit. Moreover, larger binding entities and heavy chain antibody fragments can have less than about 425 amino acids, less than about 400 amino acids, less than about 375 amino acids, less than about 350 amino acids, less than about 325 amino acids or less than about 300 amino acids if the antibody fragment is related to a heavy chain antibody subunit.

Antibodies directed against GOLPH2 can be made by any available procedure. Methods for the preparation of polyclonal antibodies are available to those skilled in the art. See, for example, Green, et al., Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1 (1992), which are hereby incorporated by reference.

Monoclonal antibodies can also be employed in the invention. The term “monoclonal antibody” as used herein refers to an antibody obtained from a population of substantially homogeneous antibodies. In other words, the individual antibodies comprising the population are identical except for occasional naturally occurring mutations in some antibodies that may be present in minor amounts. Monoclonal antibodies are highly specific, being directed against a single antigenic site. Furthermore, in contrast to polyclonal antibody preparations that typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates that the antibody is obtained from a substantially homogeneous population of antibodies, and is not to be construed as requiring production of the antibody by any particular method.

The monoclonal antibodies herein specifically include “chimeric” antibodies in which a portion of the heavy and/or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass. Fragments of such antibodies can also be used, so long as they exhibit the desired biological activity. See U.S. Pat. No. 4,816,567; Morrison et al. Proc. Natl. Acad. Sci. 81, 6851-55 (1984). In some embodiments, the constant region of the heavy and/or light chain of anti-GOLPH2 antibodies is a human sequence. In various, embodiments, the constant region of the heavy and/or light chain of anti-GOLPH2 antibodies is a sequence that does not cause an immunogenic reaction in a mammal such as a human patient.

The preparation of monoclonal antibodies is conventional and any convenient procedure can be used for making such monoclonal antibodies. See, for example, Kohler & Milstein, Nature, 256:495 (1975); Coligan, et al., sections 2.5.1-2.6.7; and Harlow, et al., in: Antibodies: A Laboratory Manual, page 726 (Cold Spring Harbor Pub. (1988)), which are hereby incorporated by reference. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG), in: Methods in Molecular Biology, Vol. 10, pages 79-104 (Humana Press (1992).

Methods of in vitro and in vivo manipulation of antibodies are available to those skilled in the art. For example, the monoclonal antibodies to be used in accordance with the present invention may be made by the hybridoma method as described above or may be made by recombinant methods, e.g., as described in U.S. Pat. No. 4,816,567. Monoclonal antibodies for use with the present invention may also be isolated from phage antibody libraries using the techniques described in Clackson et al. Nature 352: 624-628 (1991), as well as in Marks et al., J. Mol. Biol. 222: 581-597 (1991).

Methods of making antibody fragments are also known in the art (see for example, Harlow and Lane, Antibodies: A Laboratory Manual, Cold Spring Harbor Laboratory, New York, (1988), incorporated herein by reference). Antibody fragments of the present invention can be prepared by proteolytic hydrolysis of the antibody or by expression of nucleic acids encoding the antibody fragment in a suitable host. Antibody fragments can be obtained by pepsin or papain digestion of whole antibodies conventional methods. For example, antibody fragments can be produced by enzymatic cleavage of antibodies with pepsin to provide a 5S fragment described as F(ab′)₂. This fragment can be further cleaved using a thiol reducing agent, and optionally using a blocking group for the sulfhydryl groups resulting from cleavage of disulfide linkages, to produce 3.5S Fab′ monovalent fragments. Alternatively, enzymatic cleavage using pepsin produces two monovalent Fab′ fragments and an Fc fragment directly. These methods are described, for example, in U. S. Pat. Nos. 4,036,945 and 4,331,647, and references contained therein. These patents are hereby incorporated by reference in their entireties.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., Methods: a Companion to Methods in Enzymology, Vol. 2, page 97 (1991); Bird, et al., Science 242:423-426 (1988); Ladner, et al, U.S. Pat. No. 4,946,778; and Pack, et al., Bio/Technology 11:1271-77 (1993).

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., METHODS: A COMPANION TO METHODS IN ENZYMOLOGY, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details, see: Jones et al., Nature 321, 522-525 (1986); Reichmann et al., Nature 332, 323-329 (1988); Presta, Curr. Op. Struct. Biol. 2, 593-596 (1992); Holmes, et al., J. Immunol., 158:2192-2201 (1997) and Vaswani, et al., Annals Allergy, Asthma & Immunol., 81:105-115 (1998).

While standardized procedures are available to generate antibodies, the size of antibodies, the multi-stranded structure of antibodies and the complexity of six binding loops present in antibodies may constitute a hurdle to the improvement and the manufacture of large quantities of antibodies, in some embodiments. Hence, the invention further contemplates using binding entities, which comprise polypeptides that can recognize and bind to a GOLPH2 polypeptide.

A number of proteins can serve as protein scaffolds to which binding domains for GOLPH2 can be attached and thereby form a suitable binding entity. The binding domains bind or interact with GOLPH2 while the protein scaffold merely holds and stabilizes the binding domains so that they can bind. A number of protein scaffolds can be used. For example, phage capsid proteins can be used. See Review in Clackson & Wells, Trends Biotechnol. 12:173-184 (1994). Phage capsid proteins have been used as scaffolds for displaying random peptide sequences, including bovine pancreatic trypsin inhibitor (Roberts et al., PNAS 89:2429-2433 (1992)), human growth hormone (Lowman et al., Biochemistry 30:10832-10838 (1991)), Venturini et al., Protein Peptide Letters 1:70-75 (1994)), and the IgG binding domain of Streptococcus (O'Neil et al., Techniques in Protein Chemistry V (Crabb, L. ed.) pp. 517-524, Academic Press, San Diego (1994)). These scaffolds have displayed a single randomized loop or region that can be modified to include binding domains for GOLPH2.

Researchers have also used the small 74 amino acid α-amylase inhibitor Tendamistat as a presentation scaffold on the filamentous phage M13. McConnell, S. J., & Hoess, R. H., J. Mol. Biol. 250:460-470 (1995). Tendamistat is a β-sheet protein from Streptomyces tendae. It has a number of features that make it an attractive scaffold for binding peptides, including its small size, stability, and the availability of high resolution NMR and X-ray structural data. The overall topology of Tendamistat is similar to that of an immunoglobulin domain, with two β-sheets connected by a series of loops. In contrast to immunoglobulin domains, the β-sheets of Tendamistat are held together with two rather than one disulfide bond, accounting for the considerable stability of the protein. The loops of Tendamistat can serve a similar function to the CDR loops found in immunoglobulins and can be easily randomized by in vitro mutagenesis. Tendamistat is derived from Streptomyces tendae and may be antigenic in humans. Hence, binding entities that employ Tendamistat are preferably employed in vitro.

Fibronectin type III domain has also been used as a protein scaffold to which binding entities can be attached. Fibronectin type III is part of a large subfamily (Fn3 family or s-type Ig family) of the immunoglobulin superfamily. Sequences, vectors and cloning procedures for using such a fibronectin type III domain as a protein scaffold for binding entities (e.g. CDR peptides) are provided, for example, in U. S. Patent Application Publication 20020019517. See also, Bork, P. & Doolittle, R. F. (1992) Proposed acquisition of an animal protein domain by bacteria. Proc. Natl. Acad. Sci. USA 89, 8990-8994; Jones, E. Y. (1993) The immunoglobulin superfamily Curr. Opinion Struct. Biol. 3, 846-852; Bork, P., Hom, L. & Sander, C. (1994) The immunoglobulin fold. Structural classification, sequence patterns and common core. J. Mol. Biol. 242, 309-320; Campbell, I. D. & Spitzfaden, C. (1994) Building proteins with fibronectin type III modules Structure 2, 233-337; Harpez, Y. & Chothia, C. (1994).

In the immune system, specific antibodies are selected and amplified from a large library (affinity maturation). The combinatorial techniques employed in immune cells can be mimicked by mutagenesis and generation of combinatorial libraries of binding entities. Variant binding entities, antibody fragments and antibodies therefore can also be generated through display-type technologies. Such display-type technologies include, for example, phage display, retroviral display, ribosomal display, and other techniques. Techniques available in the art can be used for generating libraries of binding entities, for screening those libraries and the selected binding entities can be subjected to additional maturation, such as affinity maturation. Wright and Harris, supra., Hanes and Plucthau PNAS USA 94:4937-4942 (1997) (ribosomal display), Parmley and Smith Gene 73:305-318 (1988) (phage display), Scott TIBS 17:241-245 (1992), Cwirla et al. PNAS USA 87:6378-6382 (1990), Russel et al. Nucl. Acids Research 21:1081-1085 (1993), Hoganboom et al. Immunol. Reviews 130:43-68 (1992), Chiswell and McCafferty TIBTECH 10:80-84 (1992), and U.S. Pat. No. 5,733,743.

The invention therefore also provides methods of mutating antibodies, CDRs or binding domains to optimize their affinity, selectivity, binding strength and/or other desirable properties. A mutant binding domain refers to an amino acid sequence variant of a selected binding domain (e.g. a CDR). In general, one or more of the amino acid residues in the mutant binding domain is different from what is present in the reference binding domain. Such mutant antibodies necessarily have less than 100% sequence identity or similarity with the reference amino acid sequence. In general, mutant binding domains have at least 75% amino acid sequence identity or similarity with the amino acid sequence of the reference binding domain. Preferably, mutant binding domains have at least 80%, more preferably at least 85%, even more preferably at least 90%, and most preferably at least 95% amino acid sequence identity or similarity with the amino acid sequence of the reference binding domain.

For example, affinity maturation using phage display can be utilized as one method for generating mutant binding domains. Affinity maturation using phage display refers to a process described in Lowman et al., Biochemistry 30(45): 10832-10838 (1991), see also Hawkins et al., J. Mol. Biol. 254: 889-896 (1992). While not strictly limited to the following description, this process can be described briefly as involving mutation of several binding domains or antibody hypervariable regions at a number of different sites with the goal of generating all possible amino acid substitutions at each site. The binding domain mutants thus generated are displayed in a monovalent fashion from filamentous phage particles as fusion proteins. Fusions are generally made to the gene III product of M13. The phage expressing the various mutants can be cycled through several rounds of selection for the trait of interest, e.g. binding affinity or selectivity. The mutants of interest are isolated and sequenced.

Such methods are described in more detail in U.S. Pat. No. 5,750,373, U.S. Pat. No. 6,290,957 and Cunningham, B. C. et al., EMBO J. 13(11), 2508-2515 (1994).

Therefore, in one embodiment, the invention provides methods of manipulating binding entity or antibody polypeptides or the nucleic acids encoding them to generate binding entities, antibodies and antibody fragments with improved binding properties that recognize GOLPH2.

Such methods of mutating portions of an existing binding entity or antibody involve fusing a nucleic acid encoding a polypeptide that encodes a binding domain for GOLPH2 to a nucleic acid encoding a phage coat protein to generate a recombinant nucleic acid encoding a fusion protein, mutating the recombinant nucleic acid encoding the fusion protein to generate a mutant nucleic acid encoding a mutant fusion protein, expressing the mutant fusion protein on the surface of a phage, and selecting phage that bind to GOLPH2.

Accordingly, the invention provides antibodies, antibody fragments, and binding entity polypeptides that can recognize and bind to a GOLPH2 polypeptide. The invention further provides methods of manipulating those antibodies, antibody fragments, and binding entity polypeptides to optimize their binding properties or other desirable properties (e.g., stability, size, ease of use).

### Inhibitory Nucleic Acids

An inhibitory nucleic acid is a polymer of ribose nucleotides or deoxyribose nucleotides having more than three nucleotides in length. An inhibitory nucleic acid may include naturally-occurring nucleotides; synthetic, modified, or pseudo-nucleotides such as phosphorothiolates; as well as nucleotides having a detectable label such as ³²P, biotin, fluorescent dye or digoxigenin. An inhibitory nucleic acid that can reduce the expression and/or activity of a GOLPH2 nucleic acid may be completely complementary to the GOLPH2 nucleic acid (e.g., SEQ ID NO:16 or 18). Alternatively, some variability between the sequences may be permitted.

An inhibitory nucleic acid of the invention can hybridize to a GOLPH2 nucleic acid under intracellular conditions or under stringent hybridization conditions. The inhibitory nucleic acids of the invention are sufficiently complementary to endogenous GOLPH2 nucleic acids to inhibit expression of a GOLPH2 nucleic acid under either or both conditions. Intracellular conditions refer to conditions such as temperature, pH and salt concentrations typically found inside a cell, e.g. a mammalian cell. One example of such a mammalian cell is a cancer cell (e.g., hepatocarcinoma cell, or a myeloma cell), or any cell where GOLPH2 is or may be expressed.

Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory nucleic acids that comprise, for example, 2, 3, 4, or 5 or more stretches of contiguous nucleotides that are precisely complementary to a GOLPH2 coding sequence, each separated by a stretch of contiguous nucleotides that are not complementary to adjacent coding sequences, may inhibit the function of a GOLPH2 nucleic acid. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a ribozyme or an antisense nucleic acid molecule.

The antisense nucleic acid molecule may be single or double stranded (e.g. a small interfering RNA (siRNA)), and may function in an enzyme-dependent manner or by steric blocking. Antisense molecules that function in an enzyme-dependent manner include forms dependent on RNase H activity to degrade target mRNA. These include single-stranded DNA, RNA and phosphorothioate molecules, as well as the double-stranded RNAi/siRNA system that involves target mRNA recognition through sense-antisense strand pairing followed by degradation of the target mRNA by the RNA-induced silencing complex. Steric blocking antisense, which are RNase-H independent, interferes with gene expression or other mRNA-dependent cellular processes by binding to a target mRNA and getting in the way of other processes. Steric blocking antisense includes 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce GOLPH2 translation such that the level of GOLPH2 polypeptide is reduced. siRNAs mediate post-transcriptional gene silencing in a sequence-specific manner. See, for example, website at www.ambion.com/techlib/hottopics/rnai/rnai_may2002_print.html (last retrieved May 10, 2006). Once incorporated into an RNA-induced silencing complex, siRNA mediate cleavage of the homologous endogenous mRNA transcript by guiding the complex to the homologous mRNA transcript, which is then cleaved by the complex. The siRNA may be homologous to any region of the GOLPH2 mRNA transcript. The region of homology may be 30 nucleotides or less in length, preferable less than 25 nucleotides, and more preferably about 21 to 23 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411: 494-498 (2001); Harborth et al. Antisense Nucleic Acid Drug Dev. 13: 83-106 (2003). Typically, a target site that begin with AA, have 3′ UU overhangs for both the sense and antisense siRNA strands, and have an approximate 50% G/C content is selected. siRNAs may be chemically synthesized, created by in vitro transcription, or expressed from an siRNA expression vector or a PCR expression cassette. See, e.g., http://www.ambion.com/techlib/tb/tb_{—}506html (last retrieved May 10, 2006).

When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, preferably, 3 to 23 nucleotides in length, and may be of various nucleotide sequences including, AUG, CCC, UUCG, CCACC, CTCGAG, AAGCUU, CCACACC and UUCAAGAGA. siRNAs also may be produced in vivo by cleavage of double-stranded RNA introduced directly or via a transgene or virus. Amplification by an RNA-dependent RNA polymerase may occur in some organisms.

An antisense inhibitory nucleic acid may also be used to specifically reduce GOLPH2 expression, for example, by inhibiting transcription and/or translation. An antisense inhibitory nucleic acid is complementary to a sense nucleic acid encoding a GOLPH2. For example, it may be complementary to the coding strand of a double-stranded cDNA molecule or complementary to an mRNA sequence. It may be complementary to an entire coding strand or to only a portion thereof. It may also be complementary to all or part of the noncoding region of a nucleic acid encoding a GOLPH2. The non-coding region includes the 5′ and 3′ regions that flank the coding region, for example, the 5′ and 3′ untranslated sequences. An antisense inhibitory nucleic acid is generally at least six nucleotides in length, but may be about 8, 12, 15, 20, 25, 30, 35, 40, 45, or 50 nucleotides long. Longer inhibitory nucleic acids may also be used.

An antisense inhibitory nucleic acid may be prepared using methods known in the art, for example, by expression from an expression vector encoding the antisense inhibitory nucleic acid or from an expression cassette. Alternatively, it may be prepared by chemical synthesis using naturally-occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the antisense inhibitory nucleic acid and the sense nucleic acid.

Naturally-occurring nucleotides include the ribose or deoxyribose nucleotides adenosine, guanine, cytosine, thymine and uracil.

Examples of modified nucleotides include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylhio-N6-isopentenyladeninje, uracil-5oxyacetic acid, butoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl)uracil, (acp3)w, and 2,6-diaminopurine.

Thus, inhibitory nucleic acids of the invention may include modified nucleotides, as well as natural nucleotides such as combinations of ribose and deoxyribose nucleotides, and an antisense inhibitory nucleic acid of the invention may be of any length discussed above and that is complementary SEQ ID NO:16 and/or 18.

An inhibitor of the invention can also be a small hairpin RNA or short hairpin RNA (shRNA) is a sequence of RNA that makes a tight hairpin turn that can be used to silence gene expression via RNA interference. The shRNA hairpin structure is cleaved by the cellular machinery into a siRNA, which is then binds to and cleaves the target mRNA. shRNA can be introduced into cells via a vector encoding the shRNA, where the shRNA coding region is operably linked to a promoter. The selected promoter permits expression of the shRNA. For example, the promoter can be a U6 promoter, which is useful for continuous expression of the shRNA. The vector can, for example, be passed on to daughter cells, allowing the gene silencing to be inherited. See, McIntyre G, Fanning G, Design and cloning strategies for constructing shRNA expression vectors, BMC BIOTECUNOL. 6:1 (2006); Paddison et al., Short hairpin RNAs (shRNAs) induce sequence-specific silencing in mammalian cells, GENES DEV. 16 (8): 948-58 (2002).

An inhibitor of the invention may also be a ribozyme. A ribozyme is an RNA molecule with catalytic activity and is capable of cleaving a single-stranded nucleic acid such as an mRNA that has a homologous region. See, for example, Cech, Science 236: 1532-1539 (1987); Cech, Ann. Rev. Biochem. 59:543-568 (1990); Cech, Curr. Opin. Struct. Biol. 2: 605-609 (1992); Couture and Stinchcomb, Trends Genet. 12: 510-515 (1996). A ribozyme may be used to catalytically cleave a GOLPH2 mRNA transcript and thereby inhibit translation of the mRNA. See, for example, Haseloff et al., U.S. Pat. No. 5,641,673. A ribozyme having specificity for a GOLPH2 nucleic acid may be designed based on the nucleotide sequence of SEQ ID NO:16 and/or 18.

Methods of designing and constructing a ribozyme that can cleave an RNA molecule in trans in a highly sequence specific manner have been developed and described in the art. See, for example, Haseloff et al., Nature 334:585-591 (1988). A ribozyme may be targeted to a specific RNA by engineering a discrete “hybridization” region into the ribozyme. The hybridization region contains a sequence complementary to the target RNA that enables the ribozyme to specifically hybridize with the target. See, for example, Gerlach et al., EP 321,201. The target sequence may be a segment of about 5, 6, 7, 8, 9, 10, 12, 15, 20, or 50 contiguous nucleotides selected from a nucleotide sequence having SEQ ID NO:16 and/or 18. Longer complementary sequences may be used to increase the affinity of the hybridization sequence for the target.

The hybridizing and cleavage regions of the ribozyme can be integrally related; thus, upon hybridizing to the target RNA through the complementary regions, the catalytic region of the ribozyme can cleave the target. Thus, an existing ribozyme may be modified to target a GOLPH2 nucleic acid of the invention by modifying the hybridization region of the ribozyme to include a sequence that is complementary to the target GOLPH2 nucleic acid. Alternatively, an mRNA encoding a GOLPH2 may be used to select a catalytic RNA having a specific ribonuclease activity from a pool of RNA molecules. See, for example, Bartel & Szostak, Science 261:1411-1418 (1993).

### Methods of Identifying Inhibitors of Soluble GOLPH2

Another aspect of the invention is a method of isolating an inhibitor of soluble GOLPH2. Such a method may include: (a) contacting a cell culture comprising soluble GOLPH2 with a test agent; and (b) observing whether cells in the culture expresses IL-12 and/or interferon γ. When the cells in the culture express IL-12 and/or interferon γ, the test agent is an inhibitor of soluble GOLPH2.

In some embodiments, the test agent is an inhibitor of soluble GOLPH2 if the cells in the culture express at least 10% more IL-12 than a control consisting of a cell culture comprising soluble GOLPH2 without a test agent. In other embodiments, the test agent is an inhibitor of soluble GOLPH2 if the cells in the culture express at least 50% more IL-12 than a control consisting of a cell culture comprising soluble GOLPH2 without a test agent. In other embodiments, the test agent is an inhibitor of soluble GOLPH2 if the cells in the culture express at least two-fold more IL-12 than a control consisting of a cell culture comprising soluble GOLPH2 without a test agent. In other embodiments, the test agent is an inhibitor of soluble GOLPH2 if the cells in the culture express at least three-fold more IL-12 than a control consisting of a cell culture comprising soluble GOLPH2 without a test agent.

Examples of cells that can be used in such a method include activated monocytes, T cells, dendritic cells, B lymphoblastoid cells, antigen-presenting cells, malignant B cells, lymphoma cells and combinations thereof. In some embodiments, T cells are employed. In other embodiments, antigen presenting cells are employed. In further embodiments, dendritic cells are employed. In some embodiments, a combination of T cells and dendritic cells are employed. The cells can be activated by procedures available in the art. T cells can be stimulated with conconavalin A (ConA) before exposure to the test agent and/or the soluble GOLPH2.

In some embodiments, the test agent is an inhibitor of GOLPH2 when activated T cells express interferon γ in the presence of soluble GOLPH2. In some embodiments, dendritic cells are cultured with T cells. For example, when detecting expression of interferon γ in the presence of soluble GOLPH2 a combination of T cells and dendritic cells may be used.

The soluble GOLPH2 used in such methods can be purified, semi-purified or unpurified. In some embodiments, it may be useful to use a cell culture supernatant as the source for soluble GOLPH2. Soluble GOLPH2 is produced by a variety of cell lines, including several B cell and B cell lymphoma lines as well as hepatocellular carcinoma cell lines. For example, soluble GOLPH2 is produced by the 2E2, U266, NALM-6, REH, and RAMOS cell lines. Soluble GOLPH2 is also produced by human hepatocellular carcinoma cell line such as HepG2. The supernatants from any of these cell lines can be used as a source of GOLPH2.

The test agents can be small molecules, drugs, antibodies, inhibitory binding entities, inhibitory peptides, inhibitory nucleic acids, and combinations thereof.

### Compositions

The invention also relates to compositions containing an inhibitor of GOPLH2 such as anti-GOLPH2 antibody, or an inhibitory nucleic acid (e.g., within an expression cassette or expression vector). The compositions of the invention can be pharmaceutical compositions. In some embodiments, the compositions can include a pharmaceutically acceptable carrier. By “pharmaceutically acceptable” it is meant a carrier, diluent, excipient, and/or salt that is compatible with the other ingredients of the formulation, and not deleterious to the recipient thereof.

In some embodiments, the inhibitor is an antibody or binding entity that binds a GOLPH2 protein with a sequence such as any of SEQ ID NO: 1-15, 17, or a combination thereof. In other embodiments, the anti-GOLPH2 antibodies and binding entities preferably bind with specificity to GOLPH2 in its soluble, extracellular form. Examples of GOLPH2 polypeptide sequences to which the anti-GOLPH2 antibodies/binding entities can bind include GOLPH2 polypeptides with any of SEQ ID NO:2, 4-15. In other embodiments, the inhibitory nucleic acid is a nucleic acid that binds to a nucleic acid encoding a GOLPH2 protein with a sequence such as SEQ ID NO:16 or 18.

In some embodiments, the therapeutic agents of the invention (e.g., inhibitors of GOLPH2), are administered in a “therapeutically effective amount.” Such a therapeutically effective amount is an amount sufficient to obtain the desired physiological effect, e.g., treatment of a condition, disorder, disease and the like or reduction in symptoms of the condition, disorder, disease and the like. For example, the therapeutic agents can be administered to treat a condition, disorder, or disease such as cancer, viral infection, bacterial infection and/or microbial infection.

To achieve the desired effect(s), the GOLPH2 inhibitor and combinations thereof, may be administered as single or divided dosages. For example, GOLPH2 inhibitor(s) can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the molecule, polypeptide, antibody or nucleic acid chosen for administration, the disease, the weight, the physical condition, the health, the age of the mammal. Such factors can be readily determined by the clinician employing animal models or other test systems that are available in the art.

Administration of the therapeutic agents (e.g., inhibitors) in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the therapeutic agents and compositions of the invention may be essentially continuous over a preselected period of time or may be in a series of spaced doses. Both local and systemic administration is contemplated.

To prepare the composition, small molecules, polypeptides, nucleic acids, antibodies and other agents are synthesized or otherwise obtained, purified as necessary or desired. These small molecules, polypeptides, nucleic acids, antibodies and other agents can be suspended in a pharmaceutically acceptable carrier and/or lyophilized or otherwise stabilized. These agents can be adjusted to an appropriate concentration, and optionally combined with other agents. The absolute weight of a given small molecule, polypeptide, nucleic acid, antibody and/or other agent included in a unit dose can vary widely. For example, about 0.01 to about 2 g, or about 0.1 to about 500 mg, of at least one small molecule, polypeptide, nucleic acid, or antibody of the invention, or a plurality of small molecules, polypeptides, nucleic acids, and/or antibodies can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the therapeutic agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amount of small molecules, GOLPH2 polypeptides, inhibitory nucleic acids and/or anti-GOLPH2 antibodies for use in treatment will vary not only with the particular carrier selected but also with the route of administration, the nature of the condition being treated and the age and condition of the patient. Ultimately the attendant health care provider may determine proper dosage. In addition, a pharmaceutical composition may be formulated as a single unit dosage form.

Thus, one or more suitable unit dosage forms comprising the small molecules, GOLPH2 polypeptides, inhibitory nucleic acids and/or anti-GOLPH2 antibodies can be administered by a variety of routes including parenteral (including subcutaneous, intravenous, intramuscular and intraperitoneal), oral, rectal, dermal, transdermal, intrathoracic, intrapulmonary and intranasal (respiratory) routes. The small molecules, GOLPH2 polypeptides, inhibitory nucleic acids and/or anti-GOLPH2 antibodies may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

The compositions of the invention may be prepared in many forms that include aqueous solutions, suspensions, tablets, hard or soft gelatin capsules, and liposomes and other slow-release formulations, such as shaped polymeric gels. However, administration of small molecules, GOLPH2 polypeptides, inhibitory nucleic acids and/or anti-GOLPH2 antibodies often involves parenteral or local administration of the proteins, nucleic acids and/or antibodies in an aqueous solution or sustained release vehicle.

Thus while the small molecules, GOLPH2 polypeptides, inhibitory nucleic acids and/or anti-GOLPH2 antibodies may sometimes be administered in an oral dosage form, that oral dosage form is typically formulated such that the small molecules, GOLPH2 polypeptides, inhibitory nucleic acids and/or anti-GOLPH2 antibodies are released into the intestine after passing through the stomach. Such formulations are described in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives.

A small molecule, GOLPH2 polypeptide, inhibitory nucleic acid and/or anti-GOLPH2 antibody preparation can be formulated for parenteral administration (e.g., by injection, for example, bolus injection or continuous infusion) and may be presented in unit dosage form in ampoules, prefilled syringes, small volume infusion containers or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Suitable carriers include saline solution and other materials commonly used in the art.

The compositions can also contain other ingredients such as chemotherapeutic agents, anti-viral agents, antibacterial agents, antimicrobial agents and/or preservatives. Examples of additional therapeutic agents that may be used include, but are not limited to: alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel (Taxol®), docetaxel (Taxotere®), and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. The compounds of the invention may also be used in conjunction with radiation therapy.

The following non-limiting Examples illustrate some aspects of the development of the invention.

### EXAMPLES

The present description is further illustrated by the following examples, which should not be construed as limiting in any way. The contents of all cited references (including literature references, issued patents, published patent applications as cited throughout this application) are hereby expressly incorporated by reference.

### Example 1

### Identification of a Novel B Cell Produced Soluble Factor Acting on DC

This Example describes the identification of a soluble factor produced by B cells that has a role in directly modulating modulating interleukin 12 production in dendritic cells, and indirectly modulating T cell production of cytokines, for example, interferon gamma (IFNγ). This factor was initially termed BDSF^{IL12}; and later experiments demonstrated that BDSF^{IL12} is GOLPH2. During investigation of the mechanisms whereby B cells regulate T cell-mediated immunity, a soluble activity produced by LPS- or mitogen-activated primary B lymphocytes from human and mouse was identified. This soluble factor was also spontaneously produced by several B lymphoma cell lines that were tested, including the 2E2, U266, NALM-6, REH, and RAMOS cell lines (data not shown). This novel factor was initially designated BDSF^{IL-12} for B cell-derived soluble factor inhibiting IL-12.

### Methods

Experiments demonstrate that 2E2 cells secrete a factor into the supernatant with a molecular weight of about 80 Kda (FIG. 1B), which was termed BDSF^{IL-12}. The 2E2 cell line is a subclone of CL-01, a human monoclonal Burkett's lymphoma cell line. 2E2 cells express surface IgM and IgD, are positive for Epstein-Barr virus (EBV) and, upon induction with the CD40 ligand, IL-4, and IL-10, these cells switch to all seven downstream isotypes (Cerutti et al. J Immunol 160, 2145-57 (1998)). The following experiments were conducted to characterize this factor that is secreted by 2E2 cells.

T lymphocytes were isolated from C57BL/6 mouse spleen by CD4⁺ T cell MACS isolation kit, and were cultured for 4 days in RPMI medium (15% FBS, 20 ng/ml mIL-2). The cells were then plated at 1×10⁶ cells/well in 1 ml, and stimulated with concanavalin A (ConA) at 5 μg/ml for 24 h in the presence or absence of culture supernatant from myeloid dendritic cells (500 μl).

Dendritic cells were derived from C57BL/6 mouse bone marrow by culturing in 20% L cell conditioned medium supplemented with 20 ng/ml mIL-4 and 40 ng/ml mGM-CSF for 4 days. Dendritic cells were plated in 2 ml of medium to which BDSF^{IL-12} (1 ml of 2E2 supernatant) and/or lipopolysaccharide (LPS) (1 ug/ml) were added for 6 h. As a control, some 2 ml aliquots of dendritic cells did not receive BDSF^{IL-12} (2E2 supernatant) or LPS. After incubation for 6 h, the culture supernatant from the dendritic cells was transferred to the T cell cultures described above. IFN-γ production was measured by ELISA.

BDSF^{IL-12} strongly suppresses IFN-γ production by activated T lymphocytes, but it does so indirectly through modulating dendritic cell properties. As illustrated in FIG. 1A, non-stimulated dendritic cells did not produce detectable IFN-γ (bar 1) while LPS-stimulated dendritic cells secreted ˜20 pg/ml of IFN-γ (bar 2). Dendritic cells treated with BDSF^{IL-12} but not stimulated with LPS produced little IFN-γ (bar 3). Dendritic cells treated with BDSF^{IL-12} and stimulated with LPS (bar 4) produced little more IFN-γ than LPS-stimulated dendritic cells that received no BDSF^{IL-12} (bar 2). Non-stimulated T cells produced little IFN-γ (bars 5 and 6) but secreted significant amounts of IFN-γ when LPS-stimulated dendritic cell supernatant was added (bar 7). The amount of IFN-γ produced by non-stimulated T cells when LPS-stimulated dendritic cell supernatant was added (bar 7) was even greater than the amount of IFN-γ produced by dendritic cells stimulated with LPS (bar 2), indicating that dendritic cells produced a soluble factor(s) that stimulated resting T cells to produce IFN-γ.

When BDSF^{IL-12}-treated, LPS-stimulated dendritic cell supernatant was added to resting T cells (bar 9), IFN-γ secretion was approximately the same as that observed for dendritic cells treated with BDSF^{IL-12} and stimulated with LPS (bar 4). ConA-stimulated T cells produced ˜40 pg/ml of IFN-γ (bar 10).

However, adding LPS-stimulated dendritic cell supernatant to ConA-stimulated T cells caused a strong boost of IFN-γ secretion (bar 12), far more than the combined production of IFN-γ by LPS-stimulated dendritic cells (bar 2) and ConA-stimulated T cells (bar 10), indicating that an additional T-cell-stimulating factor may be produced by dendritic cells. This factor(s) was not present if dendritic cells were either not stimulated (bar 11), or were LPS-stimulated but also treated with BDSF^{IL-12} (bar 14). Importantly, when treating the ConA-stimulated T cells with both BDSF^{IL-12} and the LPS-stimulated dendritic cell supernatant (bar 14), the level of IFN-γ production was reduced to level of LPS-stimulated dendritic cells plus ConA-stimulated T cells (bar 2 plus bar 10), indicating that BDSF^{IL-12} does not affect T cells directly. Rather, it works via affecting the dendritic cells' ability to produce the T-cell-stimulatory activity. This conclusion is supported by the level of IFN-γ production (bar 13) where unstimulated but BDSF^{IL-12}-treated dendritic cell supernatant did not inhibit the baseline IFN-γ production by ConA-stimulated T cells (bar 10).

By a combination of biochemical and proteomic approaches aided by mass spectrometric analysis, taking advantage of a number of unique properties of BDSF^{IL-12}, the inventors identified BDSF^{IL-12} as Golgi phosphoprotein 2 (GOLPH2), from LPS-stimulated RAMOS cells (B lymphoma) (FIG. 1B).

Experiments demonstrate that BDSF^{IL-12} exhibited the following properties:
(i) It is able to selectively suppress IL-12 secretion, but not TNF-α, IL-10, IL-6 and TGF-β secretion, by activated monocytes and myeloid-derived dendritic cells in a manner independent of TGF-β, IL-10, TNF-α, and prostaglandin E2. TGF-β, IL-10, TNF-α, and prostaglandin E2 are well known inhibitors of IL-12 synthesis (Ma & Trinchieri, Adv Immunol 79, 55-92 (2001)).
(ii) BDSF^{IL-12} has little effect on other dendritic cell properties such as surface expression of CD11c, CD80, CD86, and MHC II.
(iii) Interestingly, primary B cells co-cultured with HIV-1-infected T cells produce BDSF^{IL-12} without evident infection of themselves by the virus. This indicates that the T_{H}1 impairment frequently observed in HIV-infected patients is caused, at least in part, by hyperactive B lymphocytes producing BDSF^{IL-12}.

### Example 2

### GOLPH2 is Secreted by Many Stressed and Transformed Cell Types

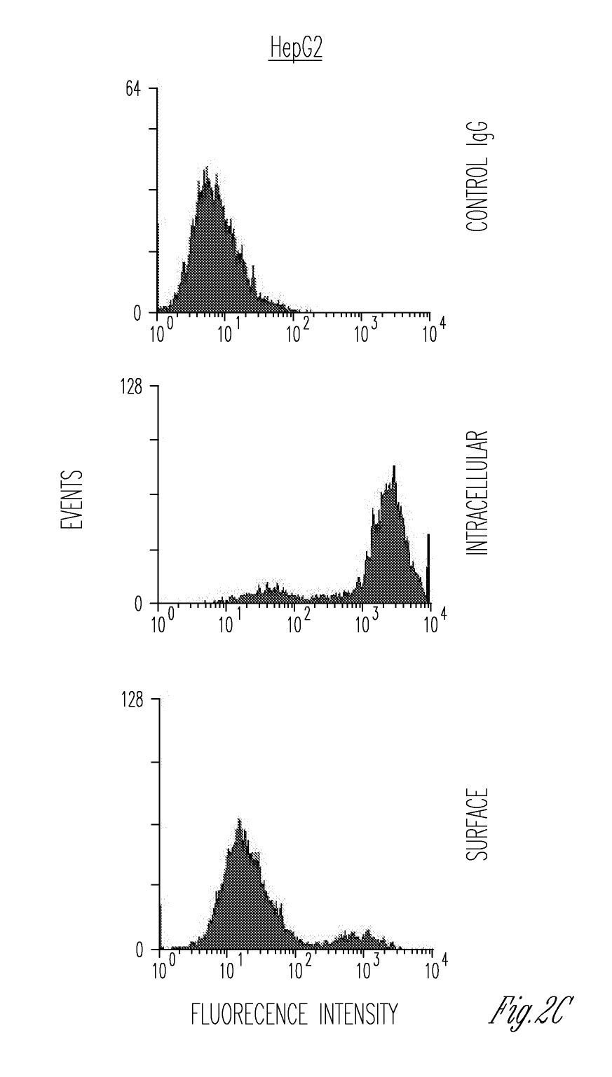

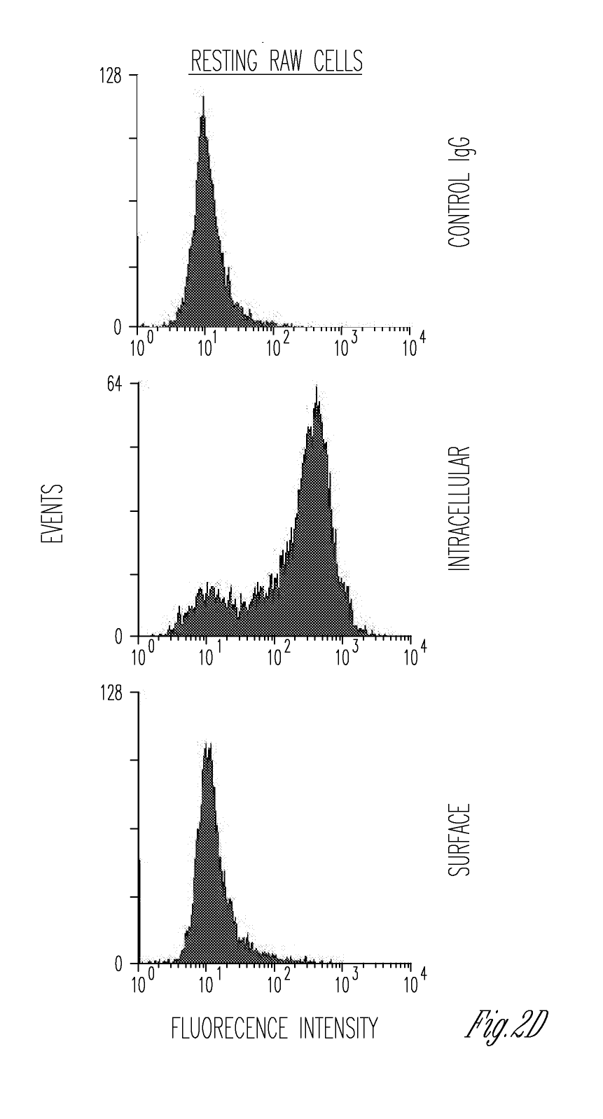

The protein expression of GOLPH2 was assessed in many cell types to better ascertain its physiological role. As shown in FIG. 2, FACS analysis indicates that the cellular location of GOLPH2 varies depending on the cell type and SDS polyacrylamide fractionation shows that GOLPH2 is secreted into the supernatant of several different cultured cell lines.

Anti-GOLPH2 antibodies for FACS and western blot analyses were obtained from Epitomics, Burlingame, Calif. (cat #: 3261-1, FIG. 2A) and from Abcam Inc., Cambridge, Mass. (cat. #ab22209; FIG. 2B). The following cell types were tested by western blot analysis: RAMOS cells (resting and LPS-activated B lymphoma cells, lanes 2-3, respectively), 2E2 cells (lane 4), HepG2 cells (human hepatocellular carcinoma (HCC), lane 5), B16 cells (mouse melanoma, lane 6), 4T1 cells (mouse mammary adenocarcinoma, lane 7), and RAW264.7 cells (mouse macrophage, lane 8). Recombinant human GOLPH2 expressed from a histidine-tagged expression vector was used as a positive control (lane 9).

FACS analysis shows that GOLPH2 is expressed abundantly intracellularly, and on the cell surface of both resting and LPS-activated primary human peripheral blood B lymphocytes (FIG. 2A). However, in the human hepatocellular carcinoma line HepG2, GOLPH2 is expressed more intracellularly than at the cell surface. In RAW264.7 cells (mouse macrophage cells), GOLPH2 expression appears entirely intracellular, and addition of LPS had little, if any, effect upon the level and locale of GOLPH2 expression.

Western blot analysis of cell culture supernatant, showed that many cancer cell types of hematopoietic and epithelial origins, both human and mouse origins, produce secreted GOLPH2 in varying amounts. The western blot in FIG. 2F shows that culture supernatants from 2E2 cells (human monoclonal Burkett's lymphoma cells, lane 4), HepG2 cells (human hepatocellular carcinoma (HCC), lane 5), B16 cells (mouse melanoma, lane 6), 4T1 cells (mouse mammary adenocarcinoma, lane 7), and RAW264.7 cells (mouse macrophage, lane 8) produce significant quantities of soluble GOLPH2. B lymphoma which cell culture supernatants contain a protein reactive with anti-GOLPH2 antibodies. Resting RAMOS B lymphoma cells produced significantly less soluble GOLPH2 while (FIG. 2B, lane 2) but more soluble GOLPH2 was produced by LPS-stimulated RAMOS B lymphoma cells (FIG. 2F, lane 3).

### Example 3

### Effect of Expression of Human GOLPH2 in Various Cells

This Example illustrates that GOLPH2 inhibits IL-12 p35 transcription and T cell IFN-γ production.

### Methods

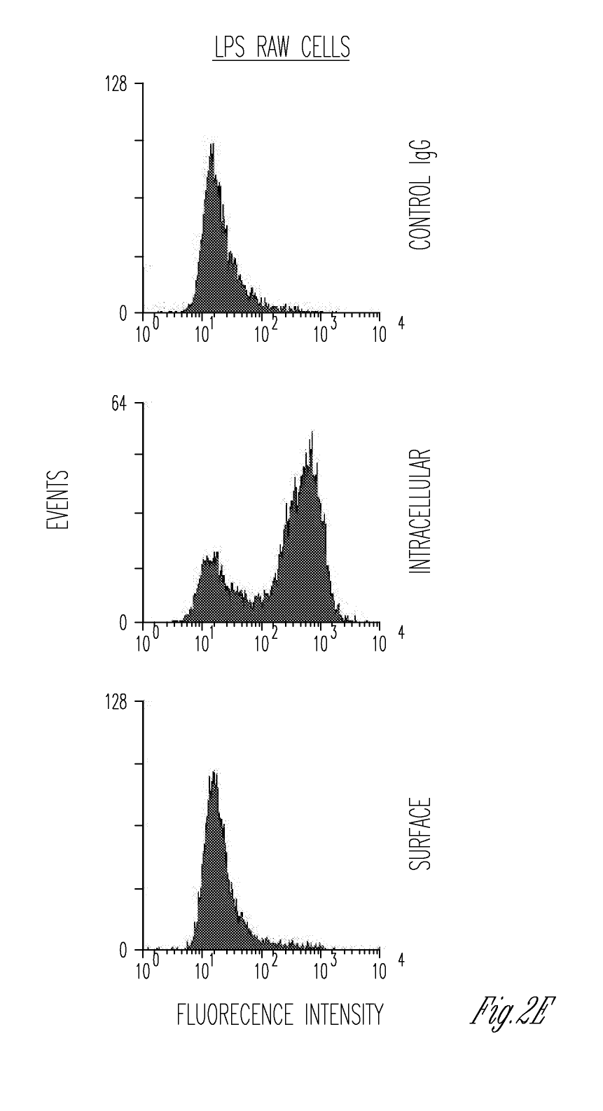

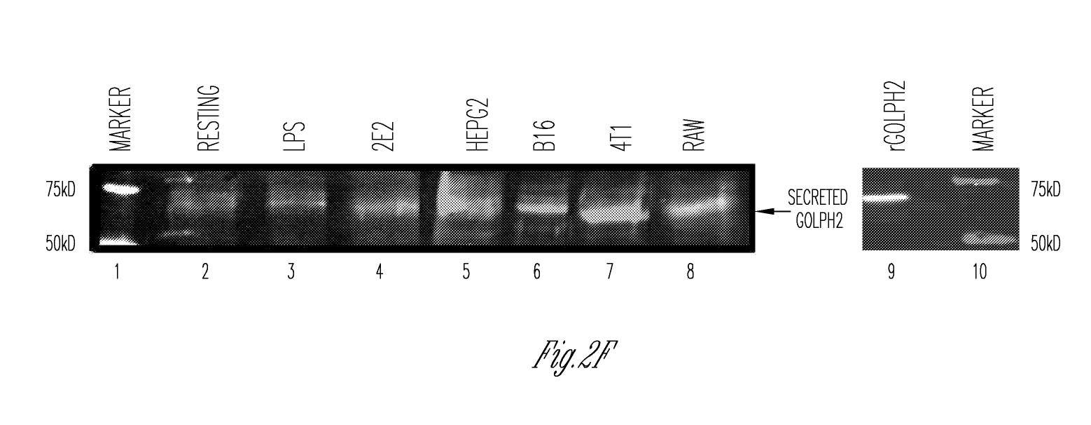

HEK293 cells were transiently transfected with a vector expressing histidine-tagged human GOLPH2, or an unrelated nuclear protein, SREBP2. Forty-eight hours after transfection, cell-free culture supernatant was collected, and the supernatant was added to co-cultures of dendritic cells and T cells as described in Example 1. IFN-γ production was measured by ELISA. The results are shown in FIG. 3A and described in more detail below.

To ascertain if GOLPH2 interacts directly or indirectly with the IL-12 promoter, a human IL-12 p35 promoter-luciferase reporter construct (see, Kim et al., Immunity 21, 643-53 (2004)) was employed. The human IL-12 p35 promoter-luciferase reporter construct was transfected into RAW264.7 cells together with one of the following effector constructs: a GOLPH2 expression vector that expressed human GOLPH2, a control empty vector (pCDNA3). Effector/reporter (E:R) molar ratios of 1:1, 2:1, and 4:1 were employed. Transfected cells were stimulated with IFN-γ (16 h) and LPS (7 h), then harvested and luciferase activity was measured from whole cell lysates. Data shown in FIG. 3B are expressed as relative promoter activity, i.e. the ratio of IFN-γ/LPS-stimulated activity over unstimulated activity.

HEK293 cells were transiently transfected with a FLAGged, empty expression vector (FLAG), or a FLAGged vector expressing human GOLPH2, or an irrelevant gene, SREBP2. Forty-eight hours after transfection, cell-free culture supernatant was collected, and 0.5 ml of the supernatant was added to 1.5 ml RAW264.7 cell transfected with either a human IL-12p35 reporter construct or a human IL-12p40 reporter construct. The cells were incubated for 6 hr. RAW264.7 cells were then stimulated with IFN-γ and LPS for 7 h before harvesting for luciferase activity measurement in triplicates. Data shown in FIG. 3C represent the mean plus standard deviation.

Another experiment was performed to test whether soluble factors within various cell supernatants could act as inhibitors of IL-12p35 and/or IL-12p40 expression. The human IL-12p35 reporter construct and the human IL-12p40 reporter construct were used in the reporter assays described above and performed in RAW264.7 cells, except that apoptotic cell (AC) or LPS-stimulated RAMOS cell culture supernatants were added to RAW264.7 cells. The cells were incubated for 6 hr. RAW264.7 cells were then stimulated with IFN-γ and LPS for 7 h before harvesting for luciferase activity measurement in triplicates. Data shown in FIG. 3D represent the mean plus standard deviation.

### Results:

When expressed heterologously in HEK293 cells, recombinant human GOLPH2 (rGOLPH2) in the culture supernatant added to mitogen-activated mouse splenic T cells suppresses IFN-γ production in a similar manner to BDSF^{IL-12}, albeit less potently (FIG. 3A, bars d and e). The rGOPLH2 was expressed as the full-length molecule, however, additional cellular mechanisms may cleave and secrete it.

The suppression by supernatants from rGOLPH2-expressing HEK293 cells was specific as an unrelated protein, sterol response element binding protein 2 (SREBP2), expressed and used in the same manner did not have any effect on IFN-γ production by T cells (FIG. 3A, bar c).

When over-expressed in the RAW264.7 macrophage cell line by co-transfection, GOLPH2 is able to inhibit IL-12p35 gene transcription dose-dependently (FIG. 3B). Culture supernatants from HEK293 cells that recombinantly express GOLPH2 strongly and selectively suppress IL-12p35 transcription but not that of p40 when added to RAW264.7 cells, (FIG. 3C upper and lower panels, respectively). This definitively confirms that GOLPH2 can act as a soluble factor, like BDSF^{IL-12}, to effect IL-12p35 gene transcription selectively. The IL-12p35 transcriptional inhibition activity in the supernatant of GOLPH2-transfected HEK293 cells was highly resistant to trypsin and boiling, just like the original BDSF^{IL-12} activity.

It is worth noting that the fact that GOLPH2-containing supernatant did not inhibit T cell-IFN-γ production as potently as did BDSF^{IL-12}-containing 2E2 supernatant suggests the existence of an additional factor(s) other than GOLPH2 in the supernatant. This notion is supported by the observation that while GOLPH2 selectively inhibits p35 transcription, BDSF^{IL-12} in LPS-activated RAMOS inhibits both p35 and p40 transcription (FIG. 3D).

### Example 4

### Blocking Extracellular GOLPH2 Reverses the Inhibition on IL-12 p35 Transcription

This Example illustrates that inhibition of extracellular BDSF^{IL-12}/GOLPH2 enhances IL-12 p35 expression. IL-12 has two subunits: a p35 subunit and a p40 subunit. As illustrated herein, BDSF^{IL-12}/GOLPH2 inhibits transcription of the IL-12 p35 subunit.

### Methods

Rabbit anti-GOLPH2 polyclonal antibodies (GP73 (N-19) were obtained from Santa Cruz Biotechnologies (Santa Cruz, Calif.), as were isotype-matched control IgG antibodies. These anti-GOLPH2 antibodies recognize a GOLPH2 protein segment with amino acids 54-90, having the following sequence (SEQ ID NO:7).

| | |
|---|---|
| 54 | RAAAERG AVELKKNEFQ GELEKQREQL DKIQSSHNFQ |

The IL-12 p35 reporter construct containing the IL-12p35 promoter operably linked to the luciferase coding region, was transfected into RAW264.7 cells. To test the effect of GOLPH2 expression on the expression levels from this IL-12p35 promoter, effector constructs including a control vector (pCR3.1), a wild type GOLPH2 (WT)-expression vector, a GOPLH2 secretion mutant R52A-expression vector, a GOLPH2 secretion-mutant R54A-expression vector, or a Roquin-expression vector was co-transfected into RAW264.7 cells with the IL-12 p35 reporter construct. The molar ratio of the effector construct to the reporter construct (E:R) was 0.2:1. A low E:R ratio (1:0.2) was used to permit the interactive (synergistic) effects between GOLPH2 and Roquin to be optimally detected. When used at higher amounts, the R52A and R54A GOLPH2 mutants were much less potent than the wild type GOLPH2 (data not shown). Luciferase activities were measured from cells following stimulation of the RAW264.7 cells with IFN-γ and LPS.

### Results

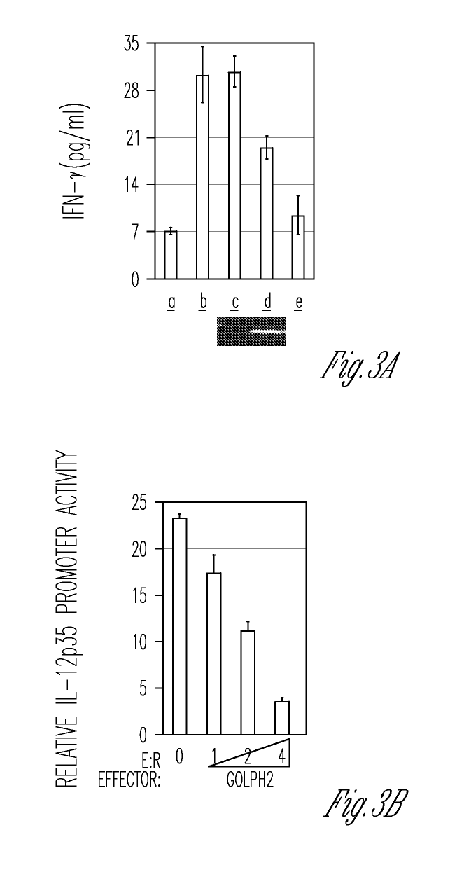

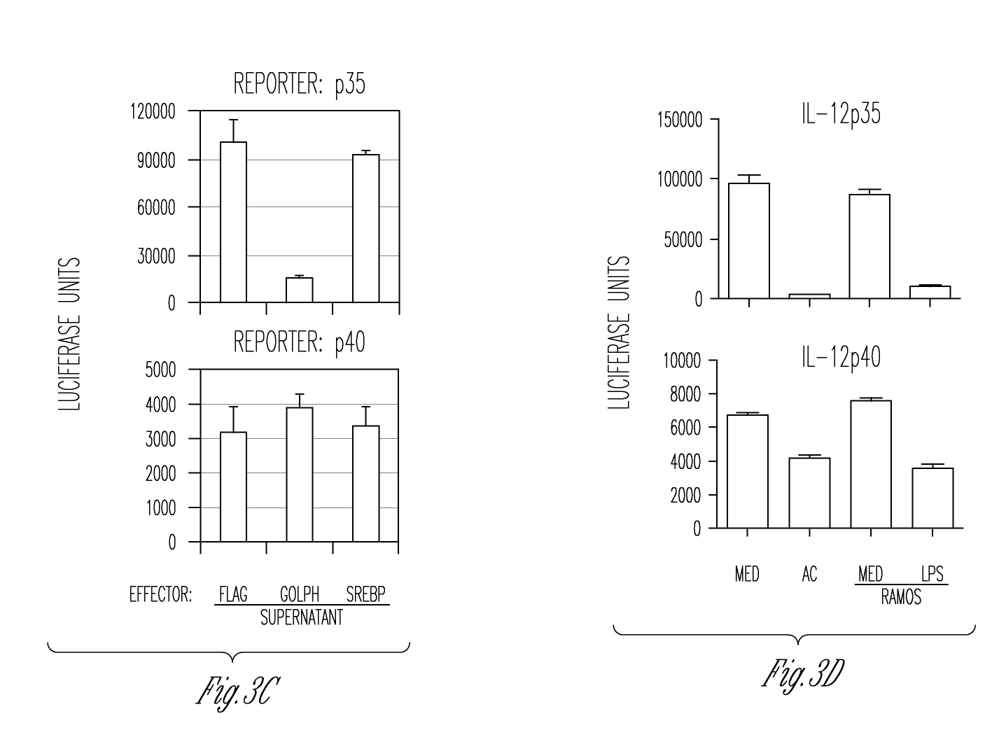

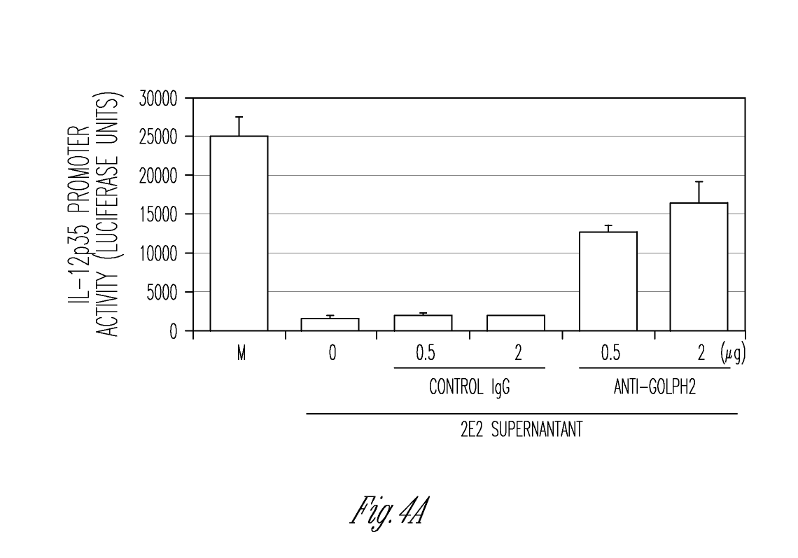

After exposure of the IL-12p35 reporter construct-containing RAW264.7 cells to IFNγ and LPS, the p35 promoter activity was greatly stimulated (FIG. 4A, Bar 1, labeled M). When the 2E2 supernatant containing soluble BDSF^{IL-12} was present, IL-12p35 promoter activity was totally suppressed (Bar 2, labeled 0), indicating that a factor (BDSF^{IL-12}) present in the supernatant was an IL-12p35 transcription inhibitor. However, when an anti-GOLPH2 antibody was present (Bars 5-6), this suppression was largely reversed. No such reversal of inhibition was observed when a control antibody was employed (Bars 3-4). These data demonstrate that BDSF^{IL-12} is an inhibitor of IL-12p35 transcription.

The fact that the anti-GOLPH2 antibody neutralized BDSF^{IL-12} inhibitory activity (FIG. 4A) also supports the conclusion that BDSF^{IL-12} and GOLPH2 share significant sequence identity, a conclusion that was further verified by mass spectrometry. Thus, BDSF^{IL-12} will be referred to as GOLPH2 in much of the disclosure.

IL-10 and TGF-β do not appear to contribute to the inhibition of IL-12 p35 transcription as determined by further neutralizing antibody experiments (data not shown), suggesting the existence of an additional, unidentified factor(s) that interact with, respond to, and/or transmit a signal provided by soluble GOLPH2. Both RAW264.7 and 2E2 cells secrete significant amounts of soluble GOLPH2 (FIG. 2F). However, antibody-mediated neutralization of GOLPH2 in RAW264.7 cells had little impact on p35 transcription (data not shown), which is in contrast to the effect of anti-GOLPH2 antibodies on p35 transcription when 2E2 supernatant was present (FIG. 4A). These suggest that there may be a second factor in the 2E2 supernatant that contributes to GOLPH2 inhibitory activity. Indeed, preliminary data indicated that the second hit in the mass spectrometry-identified proteins from LPS-activated RAMOS cell supernatant, Roquin, may act as a second factor and/or co-factor for GOLPH2, because when a Roquin expression vector is cotransfected with GOLPH2, Roquin augmented the inhibitory activity of GOLPH2 regarding p35 transcription (FIG. 4B). The augmentation by Roquin was dependent on the secretion of GOLPH2 because two secretion mutants of GOLPH2, R52A and R54A (Puri, Traffic 3, 641-53 (2002)), failed to synergistically contribute to the enhanced inhibitory activity that had been observed for wild type GOLPH2-Roquin (FIG. 4B).

Roquin was first discovered in a systematical screening of the mouse genome for autoimmune regulators, which resulted in the isolation of a mouse strain, sanroque, with severe autoimmune disease resulting from a single recessive defect in a previously unknown mechanism for repressing antibody responses to self. The sanroque mutation acts within mature T cells to cause formation of excessive numbers of follicular helper T cells and germinal centers. The mutation disrupts a repressor of ICOS, an essential co-stimulatory receptor for follicular T cells. Sanroque mice fail to repress diabetes-causing T cells, and develop high titers of auto-antibodies and pathologies consistent with lupus (Vinuesa et al., Nature 435, 452-8 (2005)). The causative mutation, M199R, is in a gene of previously unknown function, roquin (Rc3h1), which encodes a highly conserved member of the RING-type ubiquitin ligase protein family. The Roquin protein is distinguished by the presence of a CCCH zinc-finger found in RNA-binding proteins, and localization to cytosolic RNA granules implicated in regulating ICOS messenger RNA translation and stability (Yu et al., Nature 450, 299-303 (2007)).

The M199R mutant of Roquin failed to cooperate with GOLPH2 in the inhibition of IL-12 p35 transcription (data not shown), which further supports a conclusion that there is a functional interaction between GOLPH2 and Roquin.

### Example 5

### GOLPH2 Inhibits IL-12 Expression Via Activation of GC-Binding Protein

This Example shows that GOLPH2 inhibits p35 transcription targeting the same promoter element as is targeted by GC-Binding Protein and apoptotic cells engulfed by phagocytes. This promoter element is termed the “apoptotic cell response element (ACRE),” which resides between +13 and +19 of the IL-12p35 promoter and has the sequence TGCCGCG.

### Methods

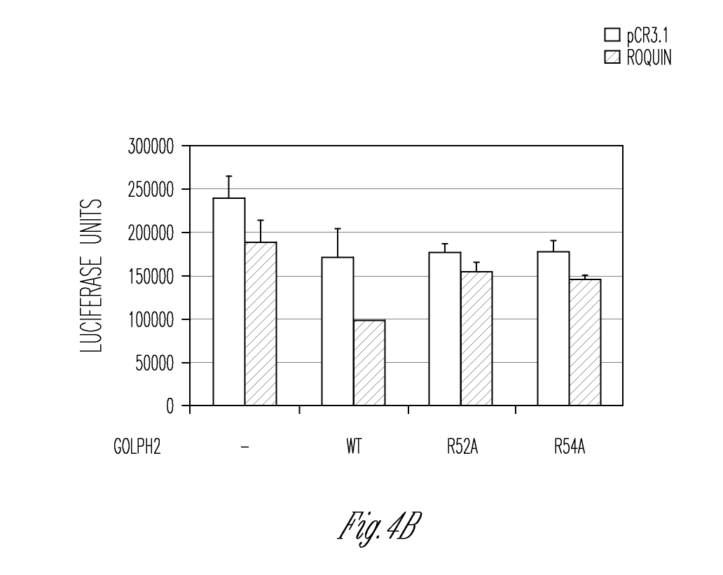

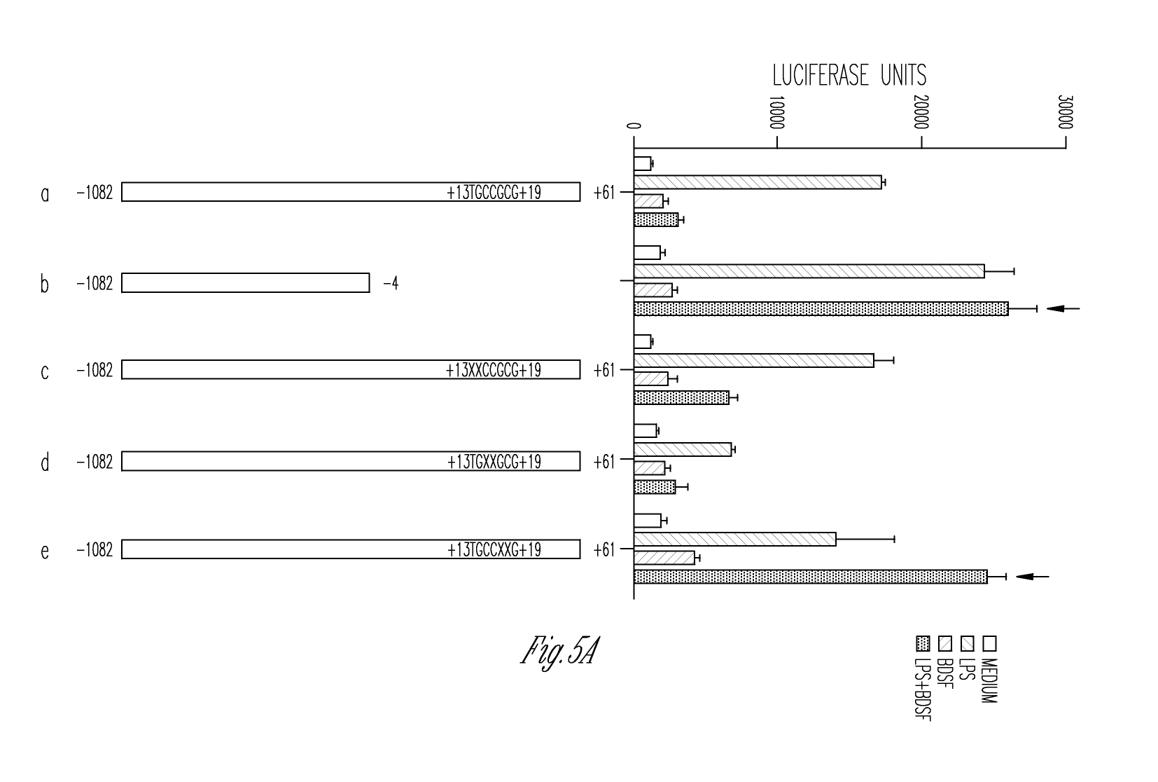

Nucleic acid segments containing wild type and mutant IL-12p35 promoter sequences spanning nucleotide positions −1082 to +61 were separately linked to a nucleic acid encoding luciferase. The wild type IL-12p35 promoter segment (a) included a TGCCGCG sequence at nucleotide positions +13 to +19. A 3′ deletion of the IL-12p35 promoter segment (b) contained only the region spanning nucleotide positions −1082 to −4. Three mutant IL-12p35 promoter segments (c-e) had specific base-substitution mutations: XXCCGCG (c), TGXXGCG (d) and TGCCXXG (e). The promoter-reporter constructs were transfected into RAW264.7 cells, and co-cultured in the presence or absence of supernatant from 2E2 cells (containing BDSF^{IL-12}). Cells were stimulated with LPS for 7 h, and luciferase activity was measured from the cell lysates. The results are shown in FIG. 5A.

RAW264.7 cells were cultured and exposed to medium (Med), or to apoptotic Jurkat cells (AC), or to supernatant from 2E2 cells (BDSF^{IL-12}) with or without IFNγ and LPS. Nuclear extracts were immunoprecipitated with anti-GC-Binding Protein antibodies (Kim et al., Immunity 21, 643-53 (2004)) followed by blotting with an anti-phospho-tyrosine mAb (pY99). Apoptotic cells (ACs) were generated by treatment with staurosporin as previously described (Kim et al., Immunity 21, 643-53 (2004)).

### Results

FIG. 5A shows that BDSF^{IL-12} selectively inhibits the transcription of the IL-12 p35 subunit gene of IL-12 primarily through the DNA motif, TGCCGCG that resides between +13 and +19 of the IL-12p35 promoter. This DNA motif is the “apoptotic cell response element (ACRE),” which was first described by the inventor in a previous study (Kim et al., Immunity 21, 643-53 (2004)). The ACRE sequence is bound by a zinc finger nuclear protein, GC-Binding Protein, which may be a factor whose activity and/or expression is activated by BDSF^{IL-12}. During phagocytosis of apoptotic cells (ACs), a novel signaling pathway is activated via the externalized phosphatidylserine (PS), resulting in tyrosine phosphorylation of the GC-Binding Protein (GC-BP), which binds directly to the IL-12p35 promoter at the ACRE site, thereby blocking the transcription (Kim et al., 2004).

FIG. 5B shows that the presence of BDSF^{IL-12} in the supernatant of cultured cells leads to activation (phosphorylation) of an approximate 80 kDa protein called GC-Binding Protein. The top panel of FIG. 5B shows a western blot of proteins from a variety of cell types that was probed with antibodies reactive with phosphorylated-GC-BP. The bottom panel of FIG. 5B shows a western blot of proteins from a variety of cell types that was probed with antibodies reactive with all GC-BP. As shown, BDSF^{IL-12} stimulates tyrosine phosphorylation of GC-Binding Protein.

BDSF^{IL-12}, like apoptotic cells (Kim et al., 2004), is therefore a potent activator of GC-BP via tyrosine phosphorylation (lanes 4 and 8, FIG. 5B). Thus, soluble BDSF^{IL-12} may inhibit expression of the IL-12p35 promoter at the ACRE site by activating GC-BP, for example, by stimulating phosphorylation of GC-BP, and the active, phosphorylated form of GC-BP then binds to, and inhibits expression from, the ACRE site on the IL-12p35 promoter.

However, BDSF^{IL-12} and apoptotic cells use different extracellular mechanisms to inhibit IL-12 expression. While apoptotic cells do so in a cell-cell contact dependent manner (Kim et al., 2004), BDSF^{IL-12} is a soluble factor that exhibits activity when it is external to the cell. Moreover, phagocytes do not produce BDSF^{IL-12} following exposure to apoptotic cells (data not shown).

These data indicate that BDSF^{IL-12}/GOLPH2 is an activator of GC-Binding Protein, whose mechanism of action is distinct from apoptotic cell activation of GC-Binding Protein. GC-Binding Protein is an inhibitory transcription factor capable of significantly reducing expression from promoters that include the TGCCGCG sequence motif, and whose inhibitory activity is activated by BDSF^{IL-12}/GOLPH2.

### Example 6

### Enhanced T_{H}1 Response in B-Cell Deficient Mice Carrying B16 Melanoma

This Example shows that IL-12 and IFN-γ production in B cell-deficient (IgM knockout) mice is significantly increased and that tumor growth in such B cell-deficient (IgM knockout) mice is significantly less than tumor growth in wild type mice. Thus, that presence of B cells can suppress anti-tumor activity.

### Methods

To implant tumors, wild type and B cell-deficient (IgM knockout) mice (five per group) were subcutaneously injected with 10⁶ tumor cells. Tumor growth was monitored periodically by measuring tumor diameters using a dial caliper. Spleens from tumor-inoculated wild type and B cell-deficient (IgM knockout) mice (five per group) were collected, and the splenocytes were cultured with tumor cells (8:1) for 7 days. The supernatants from these cultures analyzed for cytokine levels by ELISAs.

### Results

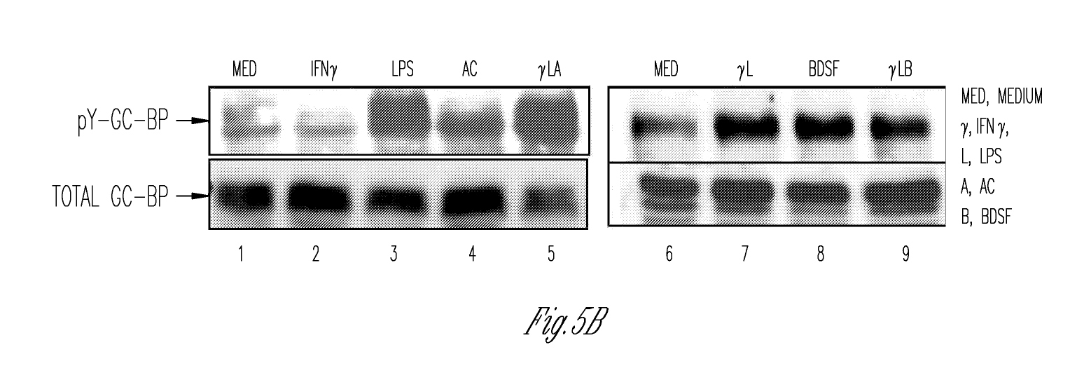

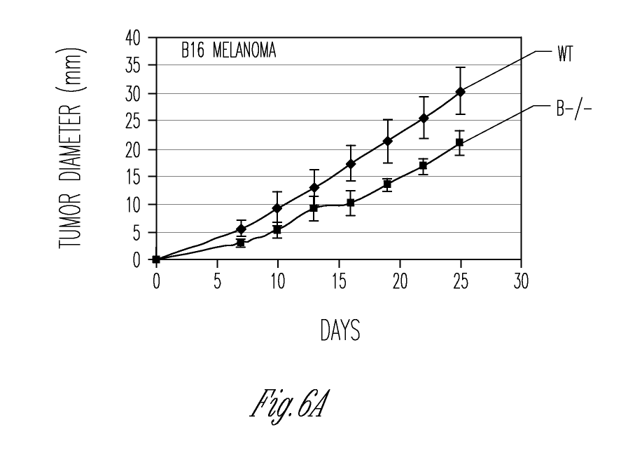

B16 melanoma growth was analyzed in wild type (WT) and B cell-deficient (IgM knockout) mice (both with a C57BL background). As shown in FIG. 6A, tumor growth in the B cell-deficient host was significantly impeded, albeit less dramatically than previously reported by Shah et al. (Int J Cancer 117, 574-86 (2005)). The impairment in B16 melanoma growth in IgM^{-/-} mice was associated with strongly increased IL-12 and IFN-γ production, as measured in the supernatant of ex vivo splenocyte-tumor co-cultures (FIG. 6B).

B cell-derived GOLPH2 may therefore suppress anti-melanoma T cell responses.

The foregoing Examples demonstrate that the BDSF^{IL-12}/GOLPH2 produced by B cells suppresses IL-12 expression. Thus, BDSF^{IL-12}/GOLPH2 may have a role in the suppression of the immune response against tumors, for example, by inhibiting IL-12 expression.

### Example 7

### Recombinant GOLPH2 Activity

This prophetic example describes experiments to confirm and further characterize that the extracellular activity of GOLPH2 alone can inhibit IL-12 expression. Purified recombinant human GOLPH2 (rGOLPH2) will be added to primary human dendritic cells (DCs), followed by stimulation with LPS to induce IL-12 production. A dose response curve will be generated to find the optimal dosage of rGOLPH2 and its duration of activity. For this purpose, a stable HEK293 cell line has been generated that overexpresses a histidine-tagged human GOLPH2 ready for medium-scale purification (FIG. 2B).

### Example 8

### GOLPH2 Induces GC-BP's Binding to ACRE In Vivo

This prophetic Example will further confirm that activated GC-BP binds to the p35 locus at the ACRE sequence in vivo by chromatin immunoprecipitation (ChIP), using procedures previously described (Kim et al. Immunity 21, 643-53 (2004)). Primary human dendritic cells will be treated rGOLPH2.

### Example 9

### RNAi-Mediated Gene Expression Silencing of GC-BP Neutralizes or Attenuates GOLPH2's Activity

This prophetic Example describes experiments designed to test whether silencing of GC-BP expression will block GOLPH2 activity, and confirm that GC-BP is a critical nuclear factor that mediates GOLPH2's inhibition of p35 transcription.

The inventor has shown that it is possible to downregulate GC-BP by

RNAi in vitro using several GC-BP-specific siRNA sequences⁶⁷. These constructs in the form of plasmid DNA have been tested in transient transfections in mouse macrophages. Sequence#3,5′-ACCUCUUGUGGCUUUGCUAdTdT-3′ (SEQ ID NO:19) has been shown to be the most effective in knocking-down GC-BP expression (>85%) (Kim et al. Immunity 21, 643-53 (2004)).

Lentiviral vectors will be utilized for introducing and expressing the specific siRNA sequence#3 described above in order to evaluate the effect of down-regulating GC-BP expression on GOLPH2's activity in primary dendritic cells. Short double-stranded siRNA template oligonucleotides under RNA Polymerase III will be introduced via lentiviral vectors. This delivery system routinely results in >80% bone marrow derived cells being positive for the transgene in long term mouse chimeras transplanted with enriched stem/progenitor cells transduced with concentrated lentivirus harboring marker genes such as enhanced green fluorescent protein (eGFP) (Rivella & Sadelain, Curr Opin Mol Ther 4, 505-14 (2002).).

### Example 10

### Identifying GOLPH2 Inhibitors

This prophetic Example describes experiments for identifying GOLPH2 inhibitors.

Previous work by the inventor has established that GC-BP is activated by a yet to be identified protein tyrosine kinase (PTK) (Kim et al. Immunity 21, 643-53 (2004)), and GC-BP may be critical for GOLPH2's activity on p35 gene transcription (FIG. 5). A panel of 156 PTK inhibitors of a wide range of receptor and non-receptor type of PTKs (EMD Chemicals Inc. Gibbstown, N.J.) will be used to identify the specific enzyme(s) important for GC-BP's activation via tyrosine phosphorylation. The specific inhibitor(s) should also reverse GOLPH2's activity. As a control, the PTK inhibitors by themselves will be tested to ascertain whether they affect the production of IL-12 by dendritic cells in the absence of GC-BP.

It is expected that GC-BP binding will increase in primary human DCs following exposure to rGOLPH2, given the strong activation (tyrosine phosphorylation) of GC-BP by BDSF^{IL-12} (FIG. 5B). It is also expected that GC-BP expression knockdown in LPS-stimulated primary human DCs by this approach will rescue IL-12 production in the presence of rGOLPH2. In order to maximize the number of cells that will express the RNAi sequence, transduced dendritic cells will be enriched by sorting with a flow cytometer for GFP expression.

GOLPH2 may be involved in posttranslational protein modification, transport of secretory proteins, cell signaling regulation, or maintenance of Golgi apparatus function. Data generated previously by the inventor using two secretion mutants of GOLPH2, R52A and R54A (Puri et al., Traffic 3, 641-53 (2002)) also indicates that GOLPH2 may function intracellularly (FIG. 4B). These potential intracellular properties of GOLPH2 may illustrate how GOLPH2 regulates IL-12 gene expression in dendritic cells. These properties will be explored further in parallel to the extracellular properties to further clarify the normal and pathological activities of GOLPH2.

### Example 11

### Identification of GOLPH2-Binding Proteins

In this prophetic Example experiments will be performed to identify the GOLPH2 receptor (GOLPH2-R). Data indicates that the IL-12 p35 transcription-inhibiting GOLPH2 is released into extracellular spaces. One likely route by which GOLPH2 exerts its actions on dendritic cells is via interaction with its membrane receptor, transducing a signal leading to the inhibition of IL-12p35 transcription and IL-12 production. Identification of the GOLPH2 receptor (GOLPH2-R) will illuminate the process by which GOLPH2 regulates DC functions at the molecular level.

### Example 12

### rGOLPH2 Binding to Dendritic Cells

In this prophetic Example, rGOLPH2 binding to cells is examined.

Human rGOLPH2 will be biotinylated using EZ-Link NHS-PEG-Biotin Reagents (PEG4 and PEG12) from Pierce. Incubation of increasing concentrations of biotinylated rGOLPH2 to 10⁶ human DCs suspended in Krebs Ringer phosphate-buffer with glucose (KRPG) will be carried out for 1 h at 4° C. followed by washing extensively with cold PBS buffer to remove excess of unbound rGOLPH2. Binding will be determined with addition of avidin conjugated with a measurable fluorophore. Specific binding will be determined as a function of time with or without addition of a 100-fold excess of unbiotinylated rGOLPH2. Plotting of maximal specific binding vs. concentration of biotinylated rGOLPH2 will reveal whether the binding is saturable. To determine reversibility of binding, dendritic cells will be incubated with a fixed amount of biotinylated rGOLPH2 first, followed by addition of increasing concentrations of unbiotinylated rGOLPH2. A dose-dependent decrease in cell-associated fluorescence in the presence of unbiotinylated rGOLPH2 will suggest that binding is reversible. Reversible and saturable binding of rGOLPH2 to dendritic cells will support the presence of a rGOLPH2 receptor(s), and we will go on to identify the binding moiety.

### Example 13

### Identification of GOLPH2 Binding Proteins by Proteomic Analysis Following GOLPH2 Pull-Down

In this prophetic Example, pull-down experiments are described to identify proteins that bind to GOLPH2.

To prevent interference from the recognition site used for pull-down with GOLPH2/target interaction, rGOLPH2 is tagged with a histidine (His)-tag. Next, a large quantity of lysates are prepared from human dendritic cells. A cocktail of protease inhibitors (Roche) will be included to prevent protein degradation during lysis. Pull-down experiments will be carried out by incubating His-tagged rGOLPH2 with a Ni-NTA solid phase affinity purification column, and washing the column extensively with PBS to remove unbound rGOLPH2. Then dendritic cell lysates will pass the rGOLPH2-bound Ni-NTA column. Extensive washing will be performed with lysate buffer followed by 20 mM immidizole in PBS. Elution will be done using a gradient of 0.2-0.5 M immidizole. All elution fractions will be separated on SDS-gel, visualized with Coomassie blue staining. Gel bands will be excised and subjected to MALDI-TOF based peptide mapping for mass determination of proteolytic fragments.

The identities of isolated GOLPH2-binding proteins in the above analysis will permit separation of membrane GOLPH2 binding proteins from cytosolic ones. Only those with one or more transmembrane domains will be further characterized for potential candidates as the putative GOLPH2 receptor(s).

### Example 14

### Identification of GOLPH2-Binding Membrane Proteins by Pull-Down

This Example describes an approach parallel to that described in Example 13 to narrow down the candidate list generated in the foregoing Examples, and to purify the membrane proteins before pull-down experiments.

First, 10⁸ THP1 cells will be incubated with a membrane-impermeable biotinylation reagent-ulfo-NHS-LC-LC-biotin (Pierce, Inc.) in Krebs-Ringer phosphate-buffer, pH 7.4, at room temperature for 30 min. The reaction will be terminated by adding glycine to a final 20 mM. Before a full-scale preparation, pilot biotinylations on a smaller scale will be carried out to search for conditions (cell density, incubation time and temperature, concentrations of biotinylating reagents) that lead to a maximal efficiency of labeling. Labeling efficiency will be checked by resolving labeled THP1 cells by SDS-PAGE and detecting biotinylated protein by Western blot with antibodies against biotin (Sigma Co.). After surface biotinylation, THP1 cells will be lysed in RIPA buffer with appropriate protease inhibitor cocktail (Roche).

Membrane fractions in the lysates will be enriched by passing the lysates through a monomeric avidin agarose column (Pierce), washed with PBS/0.6 M NaCl, reequilibrated with PBS, and eluted with 4 mM biotin in PBS. Eluted proteins will be subjected to pull-down assay described in C.1.2b using a Ni-NTA column (Qiagen) if His-tagged GOLPH2 is used. Column loading, washing and elution conditions are as described in C.1.2b. Eluted proteins will be resolved in SDS-PAGE and analyzed by

Western blot using antibody against biotin. Controls include omitting recombinant GOLPH2 in the starting materials, or using lysates from non-biotinylated THP1. Comparison among protein profiles from controls and testing the eluted fractions will help identify candidates for the GOLPH2-R.

### Example 15

### Characterization of the GOLPH2 Receptor(s)

This prophetic Example describes methods for further characterizing candidate GOLPH2 receptor proteins obtained from experiments described in the foregoing Examples.

The candidate GOLPH2 receptor proteins will be divided into two groups. To the extent antibodies are available for candidate GOLPH2-binding proteins those antibodies will be tested to ascertain whether they block GOLPH2-induced IL-12 p35 transcriptional inhibition in human dendritic cells. Such blocking will be evaluated to ascertain whether it occurs in a dose-dependent manner.

If antibodies are not available for candidate GOLPH2-binding proteins, the expression of each gene will be silenced with double strand inhibitory RNA. The impact of the gene silencing will be assessed. Scrambled sequence RNAi oligomers not corresponding to any known gene will serve as controls. If a membrane GOLPH2-binding protein is a functional GOLPH2-R, its silencing should diminish the modulating activities of GOLPH2. Dendritic cells will become more inflammatory by releasing more IL-12.

### Example 16

### Investigation of Immunological Mechanisms of B Cell-Mediated Evasion of Anti-Tumor Immunity Via GOLPH2 Using Syngeneic and Immunocompetent Mouse Tumor Models

This prophetic Example describes experiments to further investigate how B cells regulate anti-tumor CTL responses.

IgM^{-/-} B cell-deficient mice⁷² will be used to evaluate immune responses to primary syngeneic tumors. Such mice are described in Kitamura et al., Nature 350, 423-6 (1991). The primary syngeneic tumors tested will include tumors such as MC38 colon carcinoma, and B16 melanoma (all on C57BL/6 background). The ability of various agents to affect tumor growth through IL-12 expression and modulation of GOLPH2 will be tested.

In these B cell-deficient mice, several studies have shown the development of stronger anti-tumor (TS/A, MC38, EL4, 76-9 rhabdomyosarcoma, and B16) protective immunity following vaccination compared to wild type controls (Qin et al., Nat Med 4, 627-30 (1998); Perricone et al., J Immunother 27, 273-81 (2004)) and the total prevention of lung metastasis following a combination of chemokine and cytokine treatment compared to a partial response in the wild type mice (Chapoval et al., J Immunol 161, 6977-84 (1998)). Furthermore, Shah et al. showed that the increased tumor resistance in the B cell-deficient mice did not result from intrinsic changes in their non-B immunocytes because adoptive transfer of WT splenic B cells to IgM^{-/-} mice abrogated tumor rejection and led to diminished anti-tumor T_{H}1 cytokine and CTL responses (Int J Cancer 117, 574-86 (2005)). Studies involving BCR-transgenic mice indicated that B cells may inhibit anti-tumor T cell responses by antigen-nonspecific mechanisms because neither tumor-specific antibodies nor cognate T cell:B cell interactions were necessary for inhibition of tumor immunity by B cells (id.), consistent with the property of BDSF^{IL-12}/GOLPH2 being able to suppress T cell IFN-γ production indirectly through inhibiting DC-IL-12 production. Of note, relevant to the human cancer, B cell infiltration has been associated with metastatic uveal melanoma⁵³ and visceral metastatic cutaneous melanoma (Whelchel et al., Invest Ophthalmol V is Sci 34, 2603-6 (1993); Kiss et al., Pathol Oncol Res 13, 21-31 (2007); Hillen et al. Cancer Immunol Immunother 57, 97-106 (2008)).

### Example 17

### Growth of Primary Syngeneic Tumors in WT and IgM^{-/-} Mice

This prophetic Example describes experiments for testing tumor growth in wild type and B cell-deficient mice that can be exposed to different test agents. Agents can be tested to ascertain whether inhibition of GOLPH2 occurs.

Mice are injected with B16 tumor cells. Tumor growth is monitored over a three week period every three days post tumor inoculation. Tumor rejection is established by tumor-free state by day 15.

B16 is a highly aggressive and poorly immunogenic tumor. Studies indicate that with an inoculated dose of 10⁶ tumor cells, by day 15 total rejection is not achieved but tumor growth is strongly slowed down (Shah et al., Int J Cancer 117, 574-86 (2005)). To set the baseline, the growth of two histologically distinct syngeneic tumors, MC38 and B16, will be compared in WT and IgM^{-/-} mice.

TABLE 1

| ↓Tumor/ | | →Expected | Tumor | | Expected | Tumor |
|---|---|---|---|---|---|---|
| Host → | WT | growth | rejection | IgM^{-/-} → | growth | rejection |
| MC38 | 10⁶ | +++++ | — | 10⁶ | + | +++++ |
| | cells | | | cells | | |
| B16 | 10⁶ | +++++ | — | 10⁶ | + | +++ |
| | cells | | | cells | | |

### Example 18

### Anti-GOLPH2 Antibodies May Inhibit Tumor Growth

This prophetic Example describes experiments illustrating use of anti-GOLPH2 antibodies to inhibit tumor growth in wild type mice.

### Methods

Wild type and IgM^{-/-} mice (five per group) are subcutaneously injected with 10⁶ tumor cells (for example, B16 or MC38 tumor cells). Mice are then injected daily with either anti-GOLPH2 antibodies (in doses varying from 0.2 to 2 mg/kg), with isotype-matched control IgG antibodies (control) or with phosphate buffered saline (control). Anti-GOLPH2 antibodies that recognize a GOLPH2 protein segment with amino acids 54-90 (SEQ ID NO:7, shown below) may be particularly effective.

| | |
|---|---|
| 54 | RAAAERG AVELKKNEFQ GELEKQREQL DKIQSSHNFQ |

Tumor growth is monitored over a three week period every three days post tumor inoculation. Tumor rejection is established by tumor-free state by day 15.

### Results

Mice receiving anti-GOLPH2 antibodies may exhibit substantially less tumor growth over time in a dose-dependent fashion. Tumor rejection may be observed in wild type and IgM^{-/-} mice. Thus, anti-GOLPH2 antibodies may suppress anti-tumor activity.

### REFERENCES

1. Browning, M. J. & Bodmer, W. F. MHC antigens and cancer: implications for T-cell surveillance. Curr Opin Immunol 4, 613-8. (1992).
2. Chen, H., Centola, M., Altschul, S. F. & Metzger, H. Characterization of gene expression in resting and activated mast cells. J Exp Med 188, 1657-68. (1998).
3. Naor, D. Suppressor cells: permitters and promoters of malignancy? Adv Cancer Res 29, 45-125 (1979).
4. Trinchieri, G. & Scott, P. Interleukin-12: basic principles and clinical applications. Curr Top Microbiol Immunol 238, 57-78 (1999).
5. Rook, A. H. et al. Interleukin-12 therapy of cutaneous T-cell lymphoma induces lesion regression and cytotoxic T-cell responses. Blood 94, 902-8. (1999).
6. Rook, A. H. et al. The role for interleukin-12 therapy of cutaneous T cell lymphoma. Ann N Y Acad Sci 941, 177-84. (2001).
7. Ansell, S. M. et al. Phase 1 study of interleukin-12 in combination with rituximab in patients with B-cell non-Hodgkin lymphoma. Blood 99, 67-74. (2002).
8. Mortarini, R. et al. Peripheral burst of tumor-specific cytotoxic T lymphocytes and infiltration of metastatic lesions by memory CD8+ T cells in melanoma patients receiving interleukin 12. Cancer Res 60, 3559-68. (2000).
9. Gollob, J. A. et al. Phase I trial of twice-weekly intravenous interleukin 12 in patients with metastatic renal cell cancer or malignant melanoma: ability to maintain IFN-gamma induction is associated with clinical response. Clin Cancer Res 6, 1678-92. (2000).
10. Lee, P. et al. Effects of interleukin-12 on the immune response to a multipeptide vaccine for resected metastatic melanoma. J Clin Oncol 19, 3836-47. (2001).
11. Kang, W. K. et al. Interleukin 12 gene therapy of cancer by peritumoral injection of transduced autologous fibroblasts: outcome of a phase I study. Hum Gene Ther 12, 671-84. (2001).
12. Gajewski, T. F., Fallarino, F., Ashikari, A. & Sherman, M. Immunization of HLA-A2+ melanoma patients with MAGE-3 or MelanA peptide-pulsed autologous peripheral blood mononuclear cells plus recombinant human interleukin 12. Clin Cancer Res 7, 895s-901s. (2001).
13. Portielje, J. E. et al. Phase I study of subcutaneously administered recombinant human interleukin 12 in patients with advanced renal cell cancer. Clin Cancer Res 5, 3983-9. (1999).
14. Ansari, A. A. et al. Administration of recombinant rhesus interleukin-12 during acute simian immunodeficiency virus (SIV) infection leads to decreased viral loads associated with prolonged survival in SIVmac251-infected rhesus macaques. J Virol 76, 1731-43. (2002).
15. Lenzi, R. et al. Phase II study of intraperitoneal recombinant interleukin-12 (rhIL-12) in patients with peritoneal carcinomatosis (residual disease<1 cm) associated with ovarian cancer or primary peritoneal carcinoma. J Transl Med 5, 66 (2007).
16. Little, R. F. et al. Phase 2 study of pegylated liposomal doxorubicin in combination with interleukin-12 for AIDS-related Kaposi sarcoma. Blood 110, 4165-71 (2007).
17. Younes, A. et al. Phase II clinical trial of interleukin-12 in patients with relapsed and refractory non-Hodgkin's lymphoma and Hodgkin's disease. Clin Cancer Res 10, 5432-8 (2004).
18. Peterson, A. C., Harlin, H. & Gajewski, T. F. Immunization with Melan-A peptide-pulsed peripheral blood mononuclear cells plus recombinant human interleukin-12 induces clinical activity and T-cell responses in advanced melanoma. J Clin Oncol 21, 2342-8 (2003).
19. de Visser, K. E., Eichten, A. & Coussens, L. M. Paradoxical roles of the immune system during cancer development. Nat Rev Cancer 6, 24-37 (2006).
20. Andreu, P. et al. FcRgamma Activation Regulates Inflammation-Associated Squamous Carcinogenesis. Cancer Cell 17, 121-134 (2010).
21. Dalgleish, A. G. & O'Byrne, K. J. Chronic immune activation and inflammation in the pathogenesis of AIDS and cancer. Adv Cancer Res 84, 231-76 (2002).
22. Agarwal, A. et al. Flow cytometric analysis of Th1 and Th2 cytokines in PBMCs as a parameter of immunological dysfunction in patients of superficial transitional cell carcinoma of bladder. Cancer Immunol Immunother 55, 734-43 (2006).
23. Kanazawa, M. et al. Effects of PSK on T and dendritic cells differentiation in gastric or colorectal cancer patients. Anticancer Res 25, 443-9 (2005).
24. Galon, J. et al. Type, density, and location of immune cells within human colorectal tumors predict clinical outcome. Science 313, 1960-4 (2006).
25. Sheu, B. C. et al. Predominant Th2/Tc2 polarity of tumor-infiltrating lymphocytes in human cervical cancer. J Immunol 167, 2972-8 (2001).
26. Mori, T., Takada, R., Watanabe, R., Okamoto, S. & Ikeda, Y. T-helper (Th)1/Th2 imbalance in patients with previously untreated B-cell diffuse large cell lymphoma. Cancer Immunol Immunother 50, 566-8 (2001).
27. Zhang, X. L. et al. Intracellular cytokine profile of T cells from children with acute lymphoblastic leukemia. Cancer Immunol Immunother 49, 165-72 (2000).
28. Yotnda, P. et al. Analysis of T-cell defects in the specific immune response against acute lymphoblastic leukemia cells. Exp Hematol 27, 1375-83 (1999).
29. de Totero, D. et al. IL4 production and increased CD30 expression by a unique CD8+ T-cell subset in B-cell chronic lymphocytic leukaemia. Br J Haematol 104, 589-99 (1999).
30. Cui, G. et al. Distinct changes of dendritic cell number and IL-12 mRNA level in adjacent mucosa throughout the colorectal adenoma-carcinoma sequence. Cancer Immunol Immunother 56, 1993-2001 (2007).
31. Kusuda, T. et al. Relative expression levels of Th1 and Th2 cytokine mRNA are independent prognostic factors in patients with ovarian cancer. Oncol Rep 13, 1153-8 (2005).
32. Liu, Y. J. et al. Mechanism of antigen-driven selection in germinal centres. Nature 342, 929-31 (1989).
33. Rousset, F., Garcia, E. & Banchereau, J. Cytokine-induced proliferation and immunoglobulin production of human B lymphocytes triggered through their CD40 antigen. J Exp Med 173, 705-10 (1991).
34. Bennett, S. R., Carbone, F. R., Toy, T., Miller, J. F. & Heath, W. R. B cells directly tolerize CD8(+) T cells. J Exp Med 188, 1977-83 (1998).
35. Eynon, E. E. & Parker, D. C. Small B cells as antigen-presenting cells in the induction of tolerance to soluble protein antigens. J Exp Med 175, 131-8 (1992).
36. Fuchs, E. J. & Matzinger, P. B cells turn off virgin but not memory T cells. Science 258, 1156-9 (1992).
37. Parekh, V. V. et al. B cells activated by lipopolysaccharide, but not by anti-Ig and anti-CD40 antibody, induce anergy in CD8+ T cells: role of TGF-beta 1. J Immunol 170, 5897-911 (2003).
38. Skok, J., Poudrier, J. & Gray, D. Dendritic cell-derived IL-12 promotes B cell induction of Th2 differentiation: a feedback regulation of Th1 development. J Immunol 163, 4284-91 (1999).
39. Mori, L., Loetscher, H., Kakimoto, K., Bluethmann, H. & Steinmetz, M. Expression of a transgenic T cell receptor beta chain enhances collagen-induced arthritis. J Exp Med 176, 381-8 (1992).
40. Harris, D. P. et al. Reciprocal regulation of polarized cytokine production by effector B and T cells. Nat Immunol 1, 475-82 (2000).
41. Qin, Z. et al. B cells inhibit induction of T cell-dependent tumor immunity. Nat Med 4, 627-30 (1998).
42. Fillatreau, S., Sweenie, C. H., McGeachy, M. J., Gray, D. & Anderton, S. M. B cells regulate autoimmunity by provision of IL-10. Nat Immunol 3, 944-50 (2002).
43. Mauri, C., Gray, D., Mushtaq, N. & Londei, M. Prevention of arthritis by interleukin 10-producing B cells. J Exp Med 197, 489-501 (2003).
44. Matsushita, T., Yanaba, K., Bouaziz, J. D., Fujimoto, M. & Tedder, T. F.
Regulatory B cells inhibit EAE initiation in mice while other B cells promote disease progression. J Clin Invest 118, 3420-30 (2008).
45. Watanabe, R. et al. Regulatory B cells (B10 cells) have a suppressive role in murine lupus: CD19 and B10 cell deficiency exacerbates systemic autoimmunity. J Immunol 184, 4801-9 (2010).
46. Yanaba, K. et al. A regulatory B cell subset with a unique CD1dhiCDS+ phenotype controls T cell-dependent inflammatory responses. Immunity 28, 639-50 (2008).
47. Matsushita, T., Horikawa, M., Iwata, Y. & Tedder, T. F. Regulatory B cells (b10 cells) and regulatory T cells have independent roles in controlling experimental autoimmune encephalomyelitis initiation and late-phase immunopathogenesis. J Immunol 185, 2240-52 (2010).
48. Watt, V., Ronchese, F. & Ritchie, D. Resting B cells suppress tumor immunity via an MHC class-II dependent mechanism. J Immunother 30, 323-32 (2007).
49. Ma, X. & Trinchieri, G. Regulation of interleukin-12 production in antigen-presenting cells. Adv Immunol 79, 55-92 (2001).
50. Clerici, M. & Shearer, G. M. A TH1-->TH2 switch is a critical step in the etiology of HIV infection. Immunol Today 14, 107-11 (1993).
51. Cerutti, A. et al. CD40 ligand and appropriate cytokines induce switching to IgG, IgA, and IgE and coordinated germinal center and plasmacytoid phenotypic differentiation in a human monoclonal IgM+IgD+ B cell line. J Immunol 160, 2145-57 (1998).
52. Wright, L. M., Yong, S., Picken, M. M., Rockey, D. & Fimmel, C. J. Decreased survival and hepato-renal pathology in mice with C-terminally truncated GP73 (GOLPH2). Int J Clin Exp Pathol 2, 34-47 (2009).
53. Whelchel, J. C., Farah, S. E., McLean, I. W. & Burnier, M. N. Immunohistochemistry of infiltrating lymphocytes in uveal malignant melanoma. Invest Ophthalmol V is Sci 34, 2603-6 (1993).
54. Li, X., Wu, K. & Fan, D. Serum golgi phosphoprotein 2 level: a better marker than alpha-fetoprotein for diagnosing early hepatocellular carcinoma. Hepatology 50, 1682 (2009).
55. Block, T. M. et al. Use of targeted glycoproteomics to identify serum glycoproteins that correlate with liver cancer in woodchucks and humans. Proc Natl Acad Sci USA 102, 779-84 (2005).
56. Marrero, J. A. et al. GP73, a resident Golgi glycoprotein, is a novel serum marker for hepatocellular carcinoma. J Hepatol 43, 1007-12 (2005).
57. Mao, Y. et al. Golgi protein 73 (GOLPH2) is a valuable serum marker for hepatocellular carcinoma. Gut (2010).
58. Riener, M. O. et al. Golgi phosphoprotein 2 (GOLPH2) expression in liver tumors and its value as a serum marker in hepatocellular carcinomas. Hepatology 49, 1602-9 (2009).
59. Zhang, F. et al. Up-regulated Golgi phosphoprotein 2 (GOLPH2) expression in lung adenocarcinoma tissue. Clin Biochem 43, 983-91 (2010).
60. Kristiansen, G. et al. GOLPH2 protein expression as a novel tissue biomarker for prostate cancer: implications for tissue-based diagnostics. Br J Cancer 99, 939-48 (2008).
61. Kladney, R. D. et al. GP73, a novel Golgi-localized protein upregulated by viral infection. Gene 249, 53-65 (2000).
62. Clark, H. F. et al. The secreted protein discovery initiative (SPDI), a large-scale effort to identify novel human secreted and transmembrane proteins: a bioinformatics assessment. Genome Res 13, 2265-70 (2003).
63. Oh, J. J., Grosshans, D. R., Wong, S. G. & Slamon, D. J. Identification of differentially expressed genes associated with HER-2/neu overexpression in human breast cancer cells. Nucleic Acids Res 27, 4008-17 (1999).
64. Puri, S., Bachert, C., Fimmel, C. J. & Linstedt, A. D. Cycling of early Golgi proteins via the cell surface and endosomes upon lumenal pH disruption. Traffic 3, 641-53 (2002).
65. Bachert, C., Fimmel, C. & Linstedt, A. D. Endosomal trafficking and proprotein convertase cleavage of cis Golgi protein GP73 produces marker for hepatocellular carcinoma. Traffic 8, 1415-23 (2007).
66. Scott, K. L. et al. GOLPH3 modulates mTOR signalling and rapamycin sensitivity in cancer. Nature 459, 1085-90 (2009).
67. Kim, S., Elkon, K. B. & Ma, X. Transcriptional suppression of interleukin-12 gene expression following phagocytosis of apoptotic cells. Immunity 21, 643-53 (2004).
68. Vinuesa, C. G. et al. A RING-type ubiquitin ligase family member required to repress follicular helper T cells and autoimmunity. Nature 435, 452-8 (2005).
69. Yu, D. et al. Roquin represses autoimmunity by limiting inducible T-cell co-stimulator messenger RNA. Nature 450, 299-303 (2007).
70. Shah, S. et al. Increased rejection of primary tumors in mice lacking B cells: inhibition of anti-tumor CTL and TH1 cytokine responses by B cells. Int J Cancer 117, 574-86 (2005).
71. Rivella, S. & Sadelain, M. Therapeutic globin gene delivery using lentiviral vectors. Curr Opin Mol Ther 4, 505-14 (2002).
72. Kitamura, D., Roes, J., Kuhn, R. & Rajewsky, K. A B cell-deficient mouse by targeted disruption of the membrane exon of the immunoglobulin mu chain gene. Nature 350, 423-6 (1991).
73. Perricone, M. A. et al. Enhanced efficacy of melanoma vaccines in the absence of B lymphocytes. J Immunother 27, 273-81 (2004).
74. Chapoval, A. I., Fuller, J. A., Kremlev, S. G., Kamdar, S. J. & Evans, R. Combination chemotherapy and IL-15 administration induce permanent tumor regression in a mouse lung tumor model: NK and T cell-mediated effects antagonized by B cells. J Immunol 161, 6977-84 (1998).
75. Kiss, J. et al. Association of microvessel density with infiltrating cells in human cutaneous malignant melanoma. Pathol Oncol Res 13, 21-31 (2007).
76. Hillen, F. et al. Leukocyte infiltration and tumor cell plasticity are parameters of aggressiveness in primary cutaneous melanoma. Cancer Immunol Immunother 57, 97-106 (2008).
77. Shi, X., Cao, S., Mitsuhashi, M., Xiang, Z. and Ma, X. Genome-wide Analysis of Molecular Changes in Interleukin-12-induced Control of Mammary Carcinoma via Interferon-g-independent Mechanisms. Journal of Immunology In press (2004).
78. Liu, J., Xiang, Z. & Ma, X. Role of IFN regulatory factor-1 and IL-12 in immunological resistance to pathogenesis of N-methyl-N-nitrosourea-induced T lymphoma. J Immunol 173, 1184-93 (2004).
79. Linton, P. J., Harbertson, J. & Bradley, L. M. A critical role for B cells in the development of memory CD4 cells. J Immunol 165, 5558-65 (2000).
80. Ngo, V. N., Cornall, R. J. & Cyster, J. G. Splenic T zone development is B cell dependent. J Exp Med 194, 1649-60 (2001).
81. Uchida, J. et al. The innate mononuclear phagocyte network depletes B lymphocytes through Fc receptor-dependent mechanisms during anti-CD20 antibody immunotherapy. J Exp Med 199, 1659-69 (2004).
82. Xiu, Y. et al. B lymphocyte depletion by CD20 monoclonal antibody prevents diabetes in nonobese diabetic mice despite isotype-specific differences in Fc gamma R effector functions. J Immunol 180, 2863-75 (2008).
83. Hamaguchi, Y., Xiu, Y., Komura, K., Nimmerjahn, F. & Tedder, T. F. Antibody isotype-specific engagement of Fcgamma receptors regulates B lymphocyte depletion during CD20 immunotherapy. J Exp Med 203, 743-53 (2006).
84. Eisenring, M., Vom Berg, J., Kristiansen, G., Saller, E. & Becher, B. IL-12 initiates tumor rejection via lymphoid tissue-inducer cells bearing the natural cytotoxicity receptor NKp46. Nat Immunol (2010).
85. Noguchi, E., Homma, Y., Kang, X., Netea, M. G. & Ma, X. A Crohn's disease-associated NOD2 mutation suppresses transcription of human IL10 by inhibiting activity of the nuclear ribonucleoprotein hnRNP-A1. Nat Immunol 10, 471-9 (2009).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods, devices and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “an antibody” or “a nucleic acid” or “a polypeptide” includes a plurality of such antibodies, nucleic acids or polypeptides (for example, a solution of antibodies, nucleic acids or polypeptides or a series of antibody, nucleic acid or polypeptide preparations), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The Abstract is provided to comply with 37 C.F.R. §1.72(b) to allow the reader to quickly ascertain the nature and gist of the technical disclosure. The Abstract is submitted with the understanding that it will not be used to interpret or limit the scope or meaning of the claims.

The following statements of the invention are intended to describe some elements of the invention.

### STATEMENTS OF THE INVENTION

1. A method of enhancing cell-mediated immunity in a mammal in need thereof comprising administering to the mammal an inhibitor of GOLPH2 to thereby enhance cell-mediated immunity in the mammal.
2. The method of statement 1, where the inhibitor increases the mammal's endogenous production of IL-12.
3. The method of statements 1 or 2, wherein the inhibitor of GOLPH2 increases the mammal's endogenous production of interferon-γ.
4. The method of any of statements 1-3, wherein the inhibitor of GOLPH2 inhibits binding of a protein to a promoter with a sequence comprising TGCCGCG.
5. The method of statement 4, wherein the protein that binds to the promoter is a zinc finger nuclear factor.
6. The method of statement 4 or 5, wherein the protein that binds to the promoter is GC binding protein.
7. The method of any of statements 1-6, wherein the inhibitor of GOLPH2 is an antibody.
8. The method of any of statements 1-7, wherein the inhibitor of GOLPH2 is an antibody that binds specifically to GOLPH2.
9. The method of any of statements 1-8, wherein the inhibitor of GOLPH2 is an antibody that blocks GOLPH2 interaction with, or binding to, a receptor.
10. The method of any of statements 1-9, wherein the inhibitor of GOLPH2 is a monoclonal antibody.
11. The method of any of statements 1-10, wherein the inhibitor is a human antibody.
12. The method of any of statements 1-11, wherein the inhibitor is a humanized antibody.
13. The method of any of statements 1-12, wherein the inhibitor of GOLPH2 is an antibody that binds to an epitope of GOLPH2 comprising any of SEQ ID NO: 1-15, 17 or a combination thereof.
14. The method of any of statements 1-13, wherein the inhibitor of GOLPH2 is an antibody that binds to an epitope of GOLPH2 consisting essentially of any of SEQ ID NO: 1-15, 17 or a combination thereof.
15. The method of any of statements 1-14, wherein the inhibitor is an antibody that binds to a secreted form of GOLPH2.
16. The method of any of statements 1-15, wherein the inhibitor of GOLPH2 is an antibody that binds to an epitope of GOLPH2 comprising any of SEQ ID NO:2, 4-15, 17, or a combination thereof.
17. The method of any of statements 1-16, wherein the inhibitor of GOLPH2 is an antibody that binds to an epitope of GOLPH2 consisting essentially of any of SEQ ID NO:2, 4-15, 17, or a combination thereof.
18. The method of any of statements 1-6, wherein the inhibitor is an inhibitory nucleic acid.
19. The method of any of statements 1-6 or 18, wherein the inhibitor is an inhibitory nucleic acid that binds to a nucleic acid with a sequence comprising any of SEQ ID NO:16, 18 or a combination thereof.
20. The method of any of statements 1-6, 18 or 19, wherein the inhibitor is an inhibitory nucleic acid that binds to a nucleic acid with a sequence consisting essentially of any of SEQ ID NO:16, 18 or a combination thereof.
21. A method of any of statements 1-20, wherein the mammal has cancer.
22. A method of any of statements 1-21, wherein the mammal has a carcinoma, adenocarcinoma, or sarcoma.
23. The method of any of statements 1-22, where the mammal has cancer selected from the group consisting of liver cancer, lung cancer, intestinal cancer, kidney cancer, brain cancer, prostate cancer, testes cancer, ovarian cancer, breast cancer, pancreatic cancer, melanoma, lymphoma, leukemia, B-cell cancer or a combination thereof.
24. The method of any of statements 1-23, wherein the mammal has an infection.
25. The method of any of statements 1-24, wherein the mammal has a viral infection.
26. The method of any of statements 1-25, wherein the mammal has a bacterial infection.
27. The method of any of statements 1-26, wherein the mammal has an HIV or HCV infection.
28. The method of any of statements 1-27, wherein the mammal is a human.
29. A method of raising antibodies that neutralize the activity of soluble GOLPH2 comprising raising the antibodies against an peptide epitope comprising SEQ ID NO:7, or a peptide epitope analog with at least 80% sequence identity.
30. The method of statement 29, wherein the peptide epitope analog has one amino acid substitution, one added amino acid or one amino acid deletion.
31. The method of statement 29, wherein the peptide epitope analog has two amino acid substitutions, two added amino acids or two amino acid deletions.
32. The method of statement 29, wherein the peptide epitope analog has three amino acid substitutions, three added amino acids or three amino acid deletions.
33. The method of statement 29, wherein the peptide epitope analog has four amino acid substitutions, four added amino acids or four amino acid deletions.
34. A method of raising antibodies that neutralize the activity of soluble GOLPH2 comprising raising the antibodies against an peptide consisting of SEQ ID NO:7.
35. The method of any of statements 29-34, wherein the antibodies are obtained from a phage antibody library.
36. The method of any of statements 29-34, wherein the antibodies are obtained by affinity maturation.
37. The method of any of statements 29-34, wherein the peptide epitope or the peptide epitope analog is administered to an animal.
38. The method of any of statements 29-37, wherein the antibodies are humanized or human antibodies.
39. A method of isolating an inhibitor of soluble GOLPH2 comprising:

(a) contacting a cell culture comprising soluble GOLPH2 with a test agent; and

(b) observing whether cells in the culture expresses IL-12, wherein the test agent is an inhibitor of soluble GOLPH2 if the cells in the culture express IL-12.

40. The method of statement 39, wherein the cells in the culture are selected from dendritic cells, activated monocytes, T cells, cancer cells and combinations thereof.
41. The method of statement 39 or 40, wherein the test agent is an inhibitor of soluble GOLPH2 if the cells in the culture express at least 10% more IL-12 than a control consisting of a cell culture comprising soluble GOLPH2 without a test agent.
42. The method of any of statements 39-41, wherein the test agent is an inhibitor of soluble GOLPH2 if the cells in the culture express at least 50% more IL-12 than a control consisting of a cell culture comprising soluble GOLPH2 without a test agent.
43. The method of any of statements 39-42, wherein the test agent is an inhibitor of soluble GOLPH2 if the cells in the culture express at least two-fold more IL-12 than a control consisting of a cell culture comprising soluble GOLPH2 without a test agent.
44. The method of any of statements 39-43, wherein the test agent is an inhibitor of soluble GOLPH2 if the cells in the culture express at least three-fold more IL-12 than a control consisting of a cell culture comprising soluble GOLPH2 without a test agent.

What is claimed:

1. A method of enhancing cell-mediated immunity in a mammal in need thereof comprising administering to the mammal an inhibitor of GOLPH2 to thereby enhance cell-mediated immunity in the mammal.

2. The method of claim 1, where the inhibitor increases the mammal's endogenous production of IL-12.

3. The method of claim 1, wherein the inhibitor of GOLPH2 increases the mammal's endogenous production of interferon-γ.

4. The method of claim 1, wherein the inhibitor of GOLPH2 inhibits binding of a protein to a promoter with a sequence comprising TGCCGCG.

5. The method of claim 4, wherein the protein that binds to the promoter is a zinc finger nuclear factor.

6. The method of claim 4, wherein the protein that binds to the promoter is GC binding protein.

7. The method of claim 1, wherein the inhibitor of GOLPH2 is an antibody.

8. The method of claim 1, wherein the inhibitor of GOLPH2 is an antibody that binds specifically to GOLPH2.

9. The method of claim 1, wherein the inhibitor of GOLPH2 is an antibody that blocks GOLPH2 interaction with, or binding to, a receptor.

10. The method of claim 1, wherein the inhibitor of GOLPH2 is a monoclonal antibody.

11. The method of claim 1, wherein the inhibitor is a human antibody.

12. The method of claim 1, wherein the inhibitor is a humanized antibody.

13. The method of claim 1, wherein the inhibitor of GOLPH2 is an antibody that binds to an epitope of GOLPH2 comprising any of SEQ ID NO: 1-15, 17 or a combination thereof.

14. The method of claim 1, wherein the inhibitor of GOLPH2 is an antibody that binds to an epitope of GOLPH2 consisting essentially of any of SEQ ID NO: 1-15, 17 or a combination thereof.

15. The method of claim 1, wherein the inhibitor is an antibody that binds to a secreted form of GOLPH2.

16. The method of claim 1, wherein the inhibitor of GOLPH2 is an antibody that binds to an epitope of GOLPH2 comprising any of SEQ ID NO:2, 4-15, 17, or a combination thereof.

17. The method of claim 1, wherein the inhibitor of GOLPH2 is an antibody that binds to an epitope of GOLPH2 consisting essentially of any of SEQ ID NO:2, 4-15, 17, or a combination thereof.

18. The method of claim 1, wherein the inhibitor is an inhibitory nucleic acid.

19. The method of claim 1, wherein the inhibitor is an inhibitory nucleic acid that binds to a nucleic acid with a sequence comprising any of SEQ ID NO:16, 18 or a combination thereof.

20. The method of claim 1, wherein the inhibitor is an inhibitory nucleic acid that binds to a nucleic acid with a sequence consisting essentially of any of SEQ ID NO:16, 18 or a combination thereof.

21. A method of claim 1, wherein the mammal has cancer.

22. A method of claim 1, wherein the mammal has a carcinoma, adenocarcinoma, or sarcoma.

23. The method of claim 1, where the mammal has cancer selected from the group consisting of liver cancer, lung cancer, intestinal cancer, kidney cancer, brain cancer, prostate cancer, testes cancer, ovarian cancer, breast cancer, pancreatic cancer, melanoma, lymphoma, leukemia, B-cell cancer or a combination thereof.

24. The method of claim 1, wherein the mammal has an infection.

25. The method of claim 1, wherein the mammal has a viral infection.

26. The method of claim 1, wherein the mammal has a bacterial infection.

27. The method of claim 1, wherein the mammal has an HIV or HCV infection.

28. The method of claim 1, wherein the mammal is a human.
